SEQUENCE LISTING (4 sequences)

SEQ ID NO: 1 - DNA, 693 bp, Pseudomonas putida
  AlkL (1)..(693)
  CDS (1)..(693)
atgagtttttctaattataaagtaatcgcgatgccggtgttggttgctaattttgttttgggggcggccactgcatgggcgaatgaaaattatccggcgaaatctgctggctataatcagggtgactgggtcgctagcttcaatttttctaaggtctatgtgggtgaggagcttggcgatctaaatgttggagggggggctttgccaaatgctgatgtaagtattggtaatgatacaacacttacgtttgatatcgcctattttgttagctcaaatatagcggtggatttttttgttggggtgccagctagggctaaatttcaaggtgagaaatcaatctcctcgctgggaagagtcagtgaagttgattacggccctgcaattctttcgcttcaatatcattacgatagctttgagcgactttatccatatgttggggttggtgttggtcgggtgctattttttgataaaaccgacggtgctttgagttcgtttgatattaaggataaatgggcgcctgcttttcaggttggccttagatatgaccttggtaactcatggatgctaaattcagatgtgcgttatattcctttcaaaacggacgtcacaggtactcttggcccggttcctgtttctactaaaattgaggttgatcctttcattctcagtcttggtgcgtcatatgttttctaa

SEQ ID NO: 2 - protein, 230 aa, Pseudomonas putida
MSFSNYKVIAMPVLVANFVLGAATAWANENYPAKSAGYNQGDWVASFNFSKVYVGEELGDLNVGGGALPNADVSIGNDTTLTFDIAYFVSSNIAVDFFVGVPARAKFQGEKSISSLGRVSEVDYGPAILSLQYHYDSFERLYPYVGVGVGRVLFFDKTDGALSSFDIKDKWAPAFQVGLRYDLGNSWMLNSDVRYIPFKTDVTGTLGPVPVSTKIEVDPFILSLGASYVF

SEQ ID NO: 3 - DNA, 693 bp, Pseudomonas putida
  AlkL (1)..(693)
  CDS (1)..(693)
atgaatccgcctattttaaaaaaactcgctatgtcgatattagcaactagttttgtgttgggtggggccagtgcgtggtcaggtgaaatctattcgactgaaactgctggctacaatcagggcgactgggttgctagctttaatatgtctaaagtttatgtagacgagacgctaggctccctaaatgtaggtggggctactgtacccaatgctgctgtaagcatcggtaatgatacaacagtttcttttgatatttcctattttattagtaacaatgtagctttggattttttcgtcgggattccagctaaagctaagtttcaaggtgaaaaatccatctctgcgctgggaagagtcagtgaagttgattatggccctgcaattttgtcacttcagtatcattttgataattttgagcgactttatccatatgtcggactaggtgtcggtcgagtgtttttcttcgacaaaactgatggtgccttgacttcatttgatatcaaagataaatgggcgcctgctgttcaggtcggccttagatatgattttggtaactcatggatgttaaattcagatgtgcgctatattcctttcaaaacagatgtttctggtacacttggggctgcacctgtttctaccaagattgagattgatcctttcattctgagtcttggagcatcatataagttctga

SEQ ID NO: 4 - protein, 230 aa, Pseudomonas putida
MNPPILKKLAMSILATSFVLGGASAWSGEIYSTETAGYNQGDWVASFNMSKVYVDETLGSLNVGGATVPNAAVSIGNDTTVSFDISYFISNNVALDFFVGIPAKAKFQGEKSISALGRVSEVDYGPAILSLQYHFDNFERLYPYVGLGVGRVFFFDKTDGALTSFDIKDKWAPAVQVGLRYDFGNSWMLNSDVRYIPFKTDVSGTLGAAPVSTKIEIDPFILSLGASYKF

(19) United States
(12) Patent Application Publication
(10) Pub. No.: US 2018/0245107 A1
(43) Pub. Date: Aug. 30, 2018

(54) BIOCATALYTIC OXIDATION

(71) Applicant: EVONIK DEGUSSA GMBH, Essen (DE)

(72) Inventors: Thomas HAAS, Münster (DE); Simon BECK, Münster (DE); Steffen SCHAFFER, Herten (DE)

(73) Assignee: EVONIK DEGUSSA GMBH, Essen (DE)

(21) Appl. No.: 15/578,338

(22) PCT Filed: Nov. 16, 2016
(86) PCT No.: PCT/EP2016/077834

§ 371 Date: Nov. 30, 2017

(30) Foreign Application Priority Data
Nov. 17, 2015 (EP) 15194912.0

(51) Int. Cl. C12P 7/52; C12P 7/54; C12P 7/06; C12P 19/44

(52) U.S. Cl. CPC C12P 7/52; C12P 19/44; C12P 7/065; C12P 7/54

(57) ABSTRACT

There is provided a method of oxidising at least one organic substance in aerobic conditions to produce at least one alcohol, amine, acid, aldehyde, and/or ketone, the method comprising: (a) producing ethanol and/or acetate from a carbon source in aerobic conditions, comprising (i) contacting the carbon source with a reaction mixture comprising —a first acetogenic microorganism in an exponential growth phase; —free oxygen; and —a second acetogenic microorganism in a stationary phase, wherein the first and second acetogenic microorganism is capable of converting the carbon source to the acetate and/or ethanol; and (b) contacting the acetate and/or ethanol from step (a) with the organic substance and with a third microorganism capable of oxidising the organic substance to produce the alcohol, amine, acid, aldehyde, and/or ketone and wherein the acetate is a co-substrate.

### FIELD OF THE INVENTION

The present invention relates to a biocatalytic method for oxidizing at least one organic compound. In particular, the method is aerobic.

### BACKGROUND OF THE INVENTION

Oxidising organic substances is an important step in producing useful organic compounds. Alcohols, amines, acids, aldehydes, and ketones are some examples of oxidised organic compounds that are useful in our day to day life.

One method of oxidising organic substances may include the use of a metal catalyst. The oxidation catalyst used may be typically either platinum or palladium or a mixture of them, supported on a solid support material such as alumina. However, this method may be considered expensive with the presence of the catalyst and complicated.

EP100119 discloses a method of oxidizing organic compounds, such as olefins, hydrocarbons, alcohols, phenols and ketones, by means of the reaction of said substrates with hydrogen peroxide, or with a compound capable of producing hydrogen peroxide under the reaction conditions, in the presence of a titanium-silicalite. This process makes it possible for organic compounds to be oxidized with high yields and conversion rates but at a high cost due to the use of hydrogen peroxide. In particular, production of hydrogen peroxide is costly and the use of it possibly toxic. Accordingly, an alternative means of oxidising hydrocarbons that is cheaper and safer for the environment is needed.

Currently, there are also many biocatalytic methods known in the art for oxidising organic compounds. For example, EP277674 discloses a microbiological method for the terminal hydroxylation of apolar aliphatic compounds having 6 to 12 carbon atoms, such as the production of 1-octanol, by means of micro-organisms of the genus Pseudomonas putida, which are resistant to apolar phases, wherein, inter alia, a plasmid pGEc47 having the alkL gene is used, which carries the two alk operons from Pseudomonas putida as well. WO2002022845 describes a method for producing N-benzyl-4-hydroxypiperidine by hydroxylating N-benzyl-4-piperidine by E. coli cells that carry the above-mentioned plasmid pGEc47. However, in most of these methods a co-substrate such as glucose is used which makes the process of oxidizing organic substances more expensive.

Accordingly, there is a need in the art to produce a more efficient method of oxidising organic compounds that is cheaper and more environment conscious.

### DESCRIPTION OF THE INVENTION

The present invention provides a method of oxidising at least one organic compound wherein the method is a biocatalytic method that may be carried out under aerobic conditions. In particular, the method is a two-step process where acetate may be used as a co-substrate for the oxidising of the organic compound. One part involves the formation of acetate and/or ethanol from a carbon source and a further part involves the use of the acetate and/or ethanol as a co-substrate for the oxidising of at least one organic compound.

In one aspect of the present invention, there is provided a method of oxidising at least one organic substance in aerobic conditions to produce at least one alcohol, amine, acid, aldehyde, rhamnolipid and/or ketone, the method comprising:
(a) producing ethanol and/or acetate from a carbon source in aerobic conditions, comprising
   (i) contacting the carbon source with a reaction mixture comprising
      a first acetogenic microorganism in an exponential growth phase;
      free oxygen; and
      a second acetogenic microorganism in a stationary phase

wherein the first and second acetogenic microorganism is capable of converting the carbon source to the acetate and/or ethanol; and
(b) contacting the acetate and/or ethanol from step (a) with the organic substance and with a third microorganism capable of oxidising the organic substance to produce the alcohol, amine, acid, aldehyde, rhamnolipid and/or ketone and

wherein the acetate and/or ethanol is a co-substrate.

A microorganism capable of oxidising the organic substance to produce the alcohol, amine, acid, aldehyde, and/or ketone may refer to any microorganism that may be able to carry out the oxidising of the organic substance to the corresponding alcohol, amine, acid, aldehyde, rhamnolipid and/or ketone. These ‘organic compound oxidising microorganisms’ may produce the appropriate enzymes intracellularly and/or extracellularly. These organic compound oxidising microorganisms may be capable of utilising starting material for oxidising organic compounds that may be waste materials. For instance, syngas and the ethanol and/or acetate derived from syngas may be utilized for the oxidation process. This is particularly advantageous as inexpensive starting materials can be utilized that would originally have been considered waste. This also enables the removal of waste which consequently reduces environmental pollution.

In particular, the third microorganism may be any eukaryotic or prokaryotic microorganism that may be genetically modified. More in particular, the third microorganism may be a recombinant microorganism, owing to the good genetic accessibility, the microorganism may be selected from the group of bacteria, in particular Gram-negative bacteria, more in particular, the third microorganism may be a strain selected from the group consisting of Escherichia sp., Erwinia sp., Serratia sp., Providencia sp., Corynebacteria sp., Pseudomonas sp., Leptospira sp., Salmonellar sp., Brevibacteria sp., Hypomononas sp., Chromobacterium sp., Norcardia sp., fungi and yeasts. Even more in particular, the third microorganism may be selected from the group consisting of, E. coli, Pseudomonas sp., Pseudomonas fluorescens, Pseudomonas putida, Pseudomonas acidovorans, Pseudomonas aeruginosa, Acidovorax sp., Acidovorax temperans, Acinetobacter sp., Burkholderia sp., cyanobacteria, Klebsiella sp., Salmonella sp., Rhizobium sp. and Rhizobium meliloti. The third microorganism may be E. coli.

The term “acetate” as used herein, refers to both acetic acid and salts thereof, which results inevitably, because as known in the art, since the microorganisms work in an aqueous environment, there is always a balance between salt and acid present. Acetate may be used as a co-substrate in a method according to any aspect of the present invention. In particular, acetate may be present in step (b) of the method according to any aspect of the present invention at a minimum concentration of at least 10 ppm. More in particular, the acetate concentration present in step (b) may be more than or equals to 10 ppm, 20 ppm, 30 ppm, 40 ppm, 50 ppm, 100 ppm, 200 ppm, 300 ppm, 400 ppm, 500 ppm, 600 ppm, 700 ppm, 800 ppm, 900 ppm, 1000 ppm, 2000 ppm, 3000 ppm, 4000 ppm, 5000 ppm, 6000 ppm, 7000 ppm, 8000 ppm, 9000 ppm, 10000 ppm (1% wt/wt) and the like. In one example, the acetate concentration may at least be about 172 ppm. In particular, the acetate concentration may be about 160, 161, 162, 163, 164, 165, 166, 167, 168, 169, 170, 171, 172, 173, 174, 175, 176, 177, 178, 179, or 180 ppm. Even more in particular, the acetate concentration according to any aspect of the present invention may be more than or equal to 170 ppm. The acetate concentration may be less than 1750 ppm. In particular, the acetate concentration may be less than or equal to 1750, 1745, 1740, 1730, 1729, 1728, 1727, 1726, 1725, 1724, 1724, 1723, 1722, 1721, 1720 ppm. In one example, the acetate concentration according to any aspect of the present invention may be selected from the range of 150-1800, 155-1800, 160-1750, 165-1750, 170-1750, 170-1745, 170-1740, 170-1735, 170-1730, 170-1725, 170-1720 ppm. A skilled person would be capable of using any method known in the art to measure acetate concentration in an aqueous medium. For example, acetate colorimetric assay kits (Sigma-Aldrich), vacuum distillation and gas chromatography, measurements of conductivity, UV/visible spectrophotometric measurement and other methods known in the art may be used. In particular, acetate may be a co-substrate generating energy and reducing equivalents (NADH/NADPH/FADH) in the cell. The co-product of this reaction may be carbon dioxide. Carbon dioxide may be recycled in step (a) for the ethanol and/or acetate formation. The term ‘co-substrate’ used herein, refers to a substrate that may be used by a multi-substrate enzyme to carry out a reaction. For example, acetate and/or ethanol may be consumed to produce energy that may be used to reduce other co-substrates such as NAD/NADP/FAD+ to produce NADH/NADPH/FADH respectively. The ethanol and/or acetate may thus be used to maintain the ratio of NAD+/NADH, NADP+/NADPH and/or FAD+/FADH in the aqueous medium or cytosol of the cell. In particular, the reaction may be as such:

Acetyl CoA+NAD+ [symbol] NADH+H₂O+CO₂ REACTION 1

In particular, the second acetogenic microorganism in a post exponential phase may be in the stationary phase of the cell. The acetogenic cells in the log phase allow for any other acetogenic cells in the aqueous medium to produce acetate and/or ethanol in the presence of oxygen. The concentration of acetogenic cells in the log phase may be maintained in the reaction mixture. Therefore, at any point in time in the reaction, the reaction mixture comprises acetogenic cells in the log phase and acetogenic cells in another growth phase, for example in the stationary phase.

A skilled person would understand the different growth phases of microorganisms and the methods to measure them and identify them. In particular, most microorganisms in batch culture, may be found in at least four different growth phases; namely they are: lag phase (A), log phase or exponential phase (B), stationary phase (C), and death phase (D). The log phase may be further divided into the early log phase and mid to late log/exponential phase. The stationary phase may also be further distinguished into the early stationary phase and the stationary phase. For example, Cotter, J. L., 2009, Najafpour. G., 2006, Younesi, H., 2005, and Kopke, M., 2009 disclose different growth phases of acetogenic bacteria. In particular, the growth phase of cells may be measured using methods taught at least in Shuler ML, 1992 and Fuchs G., 2007.

The lag phase is the phase immediately after inoculation of the cells into a fresh medium, the population remains temporarily unchanged. Although there is no apparent cell division occurring, the cells may be growing in volume or mass, synthesizing enzymes, proteins, RNA, etc., and increasing in metabolic activity. The length of the lag phase may be dependent on a wide variety of factors including the size of the inoculum; time necessary to recover from physical damage or shock in the transfer; time required for synthesis of essential coenzymes or division factors; and time required for synthesis of new (inducible) enzymes that are necessary to metabolize the substrates present in the medium.

The exponential (log) phase of growth is a pattern of balanced growth wherein all the cells are dividing regularly by binary fission, and are growing by geometric progression. The cells divide at a constant rate depending upon the composition of the growth medium and the conditions of incubation. The rate of exponential growth of a bacterial culture is expressed as generation time, also the doubling time of the bacterial population. Generation time (G) is defined as the time (t) per generation (n=number of generations). Hence, G=t/n is the equation from which calculations of generation time derive. The exponential phase may be divided into the (i) early log phase and (ii) mid to late log/exponential phase. A skilled person may easily identify when a microorganism, particularly an acetogenic bacteria, enters the log phase. For example, the method of calculating the growth rate of acetogenic bacteria to determine if they are in the log phase mey be done using the method taught at least in Henstra A. M., 2007. In particular, the microorganism in the exponential growth phase according to any aspect of the present invention may include cells in the early log phase and mid to late log/exponential phase.

The stationary phase is the phase where exponential growth ends as exponential growth cannot be continued forever in a batch culture (e.g. a closed system such as a test tube or flask). Population growth is limited by one of three factors: 1. exhaustion of available nutrients; 2. accumulation of inhibitory metabolites or end products; 3. exhaustion of space, in this case called a lack of “biological space”. During the stationary phase, if viable cells are being counted, it cannot be determined whether some cells are dying and an equal number of cells are dividing, or the population of cells has simply stopped growing and dividing. The stationary phase, like the lag phase, is not necessarily a period of quiescence. Bacteria that produce secondary metabolites, such as antibiotics, do so during the stationary phase of the growth cycle (Secondary metabolites are defined as metabolites produced after the active stage of growth).

The death phase follows the stationary phase. During the death phase, the number of viable cells decreases geometrically (exponentially), essentially the reverse of growth during the log phase.

In one example, where O₂ is present in the reaction mixture according to any aspect of the present invention, the first acetogenic bacteria may be in an exponential growth phase and the other acetogenic bacteria may be in any other growth phase in the lifecycle of an acetogenic microorganism. In particular, according to any aspect of the present invention, the acetogenic bacteria in the reaction mixture may comprise one acetogenic bacteria in an exponential growth phase and another in the stationary phase. In the presence of oxygen, without the presence of the acetogenic bacteria in an exponential growth, the acetogenic bacteria in the stationary phase may not be capable of producing acetate and/or ethanol. This phenomenon is confirmed at least by Brioukhanov, 2006, Imlay, 2006, Lan, 2013 and the like. The inventors thus surprisingly found that in the presence of acetogenic bacteria in an exponential growth, the acetogenic bacteria in any growth phase may aerobically respire and produce acetate and/or ethanol at more than or equal to the amounts produced when the reaction mixture was absent of oxygen. In one example, the acetogenic bacteria in the exponential growth phase may be capable of removing the free oxygen from the reaction mixture, providing a suitable environment (with no free oxygen) for the acetogenic bacteria in any growth phase to metabolise the carbon substrate to produce acetate and/or ethanol.

In another example, the aqueous medium may already comprise acetogenic bacteria in any growth phase, particularly in the stationary phase, in the presence of a carbon source. In this example, there may be oxygen present in the carbon source supplied to the aqueous medium or in the aqueous medium itself. In the presence of oxygen, the acetogenic bacteria may be inactive and not produce acetate and/or ethanol prior to the addition of the acetogenic bacteria in the exponential growth phase. In this very example, the acetogenic bacteria in the exponential growth phase may be added to the aqueous medium. The inactive acetogenic bacteria already found in the aqueous medium may then be activated and may start producing acetate and/or ethanol.

In a further example, the acetogenic bacteria in any growth phase may be first mixed with the acetogenic bacteria in the exponential growth phase and then the carbon source and/or oxygen added.

According to any aspect of the present invention, a microorganism in the exponential growth phase grown in the presence of oxygen may result in the microorganism gaining an adaptation to grow and metabolise in the presence of oxygen. In particular, the microorganism may be capable of removing the oxygen from the environment surrounding the microorganism. This newly acquired adaptation allows for the acetogenic bacteria in the exponential growth phase to rid the environment of oxygen and therefore produce acetate and ethanol from the carbon source. In particular, the acetogenic bacteria with the newly acquired adaptation allows for the bacteria to convert the carbon source to acetate and/or ethanol.

In one example, the acetogenic bacteria in the reaction mixture according to any aspect of the present impression may comprise a combination of cells: cells in the log phase and cells in the stationary phase. In the method according to any aspect of the present invention the acetogenic cells in the log phase may comprise a growing rate selected from the group consisting of 0.01 to 2 h⁻¹, 0.01 to 1 h⁻¹, 0.05 to 1 h⁻¹, 0.05 to 2 h⁻¹ 0.05 to 0.5 h⁻¹ and the like. In one example, the OD₆₀₀ of the cells of the log phase acetogenic cells in the reaction mixture may be selected from the range consisting of 0.001 to 2, 0.01 to 2, 0.1 to 1, 0.1 to 0.5 and the like. A skilled person would be able to use any method known in the art to measure the OD₆₀₀ and determine the growth rate of the cells in the reaction mixture and/or to be added in the reaction mixture. For example, Koch (1994) may be used. In particular, bacterial growth can be determined and monitored using different methods. One of the most common is a turbidity measurement, which relies upon the optical density (OD) of bacteria in suspension and uses a spectrophotometer. The OD may be measured at 600 nm using a UV spectrometer.

In order to maintain the concentration of the first and second acetogenic bacteria in the reaction mixture, a skilled person may be capable of extracting a sample at fixed time points to measure the OD₆₀₀, pH, concentration of oxygen and concentration of ethanol and/or higher alcohols formed. The skilled person would then be able to add the necessary component(s) to maintain the concentration of first and second acetogenic bacteria in the reaction mixture and to ensure an optimum environment is maintained for the production of ethanol and/or acetate.

The term “acetogenic bacteria” as used herein refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), Clostridium ljungdahlii ERI-2 (ATCC 55380), Clostridium ljungdahlii 0-52 (ATCC 55989), Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui). More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium ljungdahlii. In particular, strains selected from the group consisting of Clostridium ljungdahlii PETC, Clostridium ljungdahlii ER12, Clostridium ljungdahlii COL and Clostridium ljungdahlii 0-52 may be used in the conversion of synthesis gas to hexanoic acid. These strains for example are described in WO 98/00558, WO 00/68407, ATCC 49587, ATCC 55988 and ATCC 55989. The first and second acetogenic bacteria used according to any aspect of the present invention may be the same or different bacteria. For example, in one reaction mixture the first acetogenic bacteria may be Clostridium ljungdahlii in the log phase and the second acetogenic bacteria may be Clostridium ljungdahlii in the stationary phase. In another example, in the reaction mixture the first acetogenic bacteria may be Clostridium ljungdahlii in the log phase and the second acetogenic bacteria may be Clostridium carboxidivorans in the stationary phase.

The phrase ‘the genetically modified cell has an increased activity, in comparison with its wild type, in enzymes’ as used herein refers to the activity of the respective enzyme that is increased by a factor of at least 2, in particular of at least 10, more in particular of at least 100, yet more in particular of at least 1000 and even more in particular of at least 10000.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures. Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. Similarly, a decreased activity of an enzyme refers to decreased intracellular activity. In one example, the increased expression of an enzyme according to any aspect of the present invention may be 5, 10, 15, 20, 25, 30, 25, 40, 45, 50, 55, 60, 65, 70, 75, 80, 85, 90, 95 or 100% more relative to the expression of the enzyme in the wild type cell. Similarly, the decreased expression of an enzyme according to any aspect of the present invention may be 5, 10, 15, 20, 25, 30, 25, 40, 45, 50, 55, 60, 65, 70, 75, 80, 85, 90, 95 or 100% less relative to the expression of the enzyme in the wild type cell.

In the reaction mixture according to any aspect of the present invention, there may be oxygen present. It is advantageous to incorporate O₂ in the reaction mixture and/or gas flow being supplied to the reaction mixture as most waste gases including synthesis gas comprises oxygen in small or large amounts. It is difficult and costly to remove this oxygen prior to using synthesis gas as a carbon source for production of higher alcohols. The method according to any aspect of the present invention allows the production of at least one higher alcohol without the need to first remove any trace of oxygen from the carbon source. This allows for time and money to be saved.

More in particular, the O₂ concentration in the gas flow may be may be present at less than 1% by volume of the total amount of gas in the gas flow. In particular, the oxygen may be present at a concentration range of 0.000005 to 2% by volume, at a range of 0.00005 to 2% by volume, 0.0005 to 2% by volume, 0.005 to 2% by volume, 0.05 to 2% by volume, 0.00005 to 1.5% by volume, 0.0005 to 1.5% by volume, 0.005 to 1.5% by volume, 0.05 to 1.5% by volume, 0.5 to 1.5% by volume, 0.00005 to 1% by volume, 0.0005 to 1% by volume, 0.005 to 1% by volume, 0.05 to 1% by volume, 0.5 to 1% by volume, 0.55 to 1% by volume, 0.60 to 1% by volume, particularly at a range of 0.60 to 1.5%, 0.65 to 1%, and 0.70 to 1% by volume in the gas phase of the gas flow and/or in the medium. In particular, the acetogenic microorganism is particularly suitable when the proportion of O₂ in the gas phase/flow is about 0.00005, 0.0005, 0.005, 0.05, 0.5, 0.6, 0.7, 0.8, 0.9, 1, 1.5, 2% by volume in relation to the volume of the gas in the gas flow. In one example, the level of oxygen is 0.5 parts per million (ppm) in the gas phase of the environment to which the microorganisms (first, second and/or third) are exposed to. A skilled person would be able to use any one of the methods known in the art to measure the volume concentration of oxygen in the gas flow. In particular, the volume of oxygen may be measured using any method known in the art. In one example, a gas phase concentration of oxygen may be measured by a trace oxygen dipping probe from PreSens Precision Sensing GmbH. Oxygen concentration may be measured by fluorescence quenching, where the degree of quenching correlates to the partial pressure of oxygen in the gas phase. Even more in particular, the first and second microorganisms according to any aspect of the present invention are capable of working optimally in the aqueous medium when the oxygen is supplied by a gas flow with concentration of oxygen of less than 1% by volume of the total gas, in about 0.015% by volume of the total volume of gas in the gas flow supplied to the reaction mixture.

According to any aspect of the present invention, the aerobic conditions in which the carbon source is converted to ethanol and/or acetate in the reaction mixture refers to gas surrounding the reaction mixture. The gas may comprise at least about 0.00005% to about 1% by volume of the total gas of oxygen and other gases including carbon sources such as CO, CO₂ and the like.

The aqueous medium according to any aspect of the present invention may comprise oxygen. The oxygen may be dissolved in the medium by any means known in the art. In particular, the oxygen may be present at 0.5 mg/L in the absence of cells. In particular, the dissolved concentration of free oxygen in the aqueous medium may at least be 0.01 mg/L. In another example, the dissolved oxygen may be about 0.01, 0.02, 0.03, 0.04, 0.05, 0.1, 0.2, 0.3, 0.4, 0.5 mg/L. In particular, the dissolved oxygen concentration may be 0.01-0.5 mg/L, 0.01-0.4 mg/L, 0.01-0.3 mg/L, 0.01-0.1 mg/L. In particular, the oxygen may be provided to the aqueous medium in a continuous gas flow. More in particular, the aqueous medium may comprise oxygen and a carbon source comprising CO and/or CO₂. More in particular, the oxygen and a carbon source comprising CO and/or CO₂ is provided to the aqueous medium in a continuous gas flow. Even more in particular, the continuous gas flow comprises synthesis gas and oxygen. In one example, both gases are part of the same flow/stream. In another example, each gas is a separate flow/stream provided to the aqueous medium. These gases may be divided for example using separate nozzles that open up into the aqueous medium, frits, membranes within the pipe supplying the gas into the aqueous medium and the like. The oxygen may be free oxygen. According to any aspect of the present invention, ‘a reaction mixture comprising free oxygen’ refers to the reaction mixture comprising elemental oxygen in the form of O₂. The O₂ may be dissolved oxygen in the reaction mixture. In particular, the dissolved oxygen may be in the concentration of 5 ppm (0.000005% vol; 5×10⁻⁶). A skilled person may be capable of using any method known in the art to measure the concentration of dissolved oxygen. In one example, the dissolved oxygen may be measured by Oxygen Dipping Probes (Type PSt6 from PreSens Precision Sensing GmbH, Regensburg, Germany.

According to any aspect of the present invention, the first, second and/or third microorganism may be a genetically modified microorganism. The genetically modified cell or microorganism may be genetically different from the wild type cell or microorganism. The genetic difference between the genetically modified microorganism according to any aspect of the present invention and the wild type microorganism may be in the presence of a complete gene, amino acid, nucleotide etc. in the genetically modified microorganism that may be absent in the wild type microorganism. In one example, the genetically modified microorganism according to any aspect of the present invention may comprise enzymes that enable the microorganism to produce at least one amino acid. The wild type microorganism relative to the genetically modified microorganism according to any aspect of the present invention may have none or no detectable activity of the enzymes that enable the genetically modified microorganism to produce at least one amino acid. As used herein, the term ‘genetically modified microorganism’ may be used interchangeably with the term ‘genetically modified cell’. The genetic modification according to any aspect of the present invention may be carried out on the cell of the microorganism.

The phrase “wild type” as used herein in conjunction with a cell or microorganism may denote a cell with a genome make-up that is in a form as seen naturally in the wild. The term may be applicable for both the whole cell and for individual genes. The term “wild type” therefore does not include such cells or such genes where the gene sequences have been altered at least partially by man using recombinant methods.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least twice, especially at least 10 times, at least 100 times, at least 1000 times or at least 10000 times more amino acid than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (amino acid) in the nutrient medium.

The term “second microorganism” or “third microorganism”, refers to a microorganism that may be different from “the first microorganism” according to any aspect of the present invention. The culture medium to be used must be suitable for the requirements of the particular strains. Descriptions of culture media for various microorganisms are given in “Manual of Methods for General Bacteriology”.

All percentages (%) are, unless otherwise specified, mass percent.

With respect to the source of substrates comprising carbon dioxide and/or carbon monoxide, a skilled person would understand that many possible sources for the provision of CO and/or CO₂ as a carbon source exist. It can be seen that in practice, as the carbon source of the present invention any gas or any gas mixture can be used which is able to supply the microorganisms with sufficient amounts of carbon, so that acetate and/or ethanol, may be formed from the source of CO and/or CO₂.

Generally for the cell of the present invention the carbon source comprises at least 50% by weight, at least 70% by weight, particularly at least 90% by weight of CO₂ and/or CO, wherein the percentages by weight—% relate to all carbon sources that are available to the cell according to any aspect of the present invention. The carbon material source may be provided.

Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the mixed culture of the present invention.

According to any aspect of the present invention, the carbon source may be synthesis gas. Synthesis gas can for example be produced as a by-product of coal gasification. Accordingly, the microorganism according to any aspect of the present invention may be capable of converting a substance which is a waste product into a valuable resource.

In another example, synthesis gas may be a by-product of gasification of widely available, low-cost agricultural raw materials for use with the mixed culture of the present invention to produce substituted and unsubstituted organic compounds.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Usually, a portion of the synthesis gas obtained from the gasification process is first processed in order to optimize product yields, and to avoid formation of tar. Cracking of the undesired tar and CO in the synthesis gas may be carried out using lime and/or dolomite. These processes are described in detail in for example, Reed, 1981.

Mixtures of sources can be used as a carbon source.

According to any aspect of the present invention, a reducing agent, for example hydrogen may be supplied together with the carbon source. In particular, this hydrogen may be supplied when the C and/or CO₂ is supplied and/or used. In one example, the hydrogen gas is part of the synthesis gas present according to any aspect of the present invention. In another example, where the hydrogen gas in the synthesis gas is insufficient for the method of the present invention, additional hydrogen gas may be supplied.

In another example, carbon dioxide may be produced in Reaction I as mentioned above. The carbon dioxide may then be recycled in step (a) according to any aspect of the present invention to produce acetate and/or ethanol. There may thus be no waste of side products produced according to any aspect of the present invention. No carbon source may be needed to be added and/or topped up in step (a) to carry out the method according to any aspect of the present invention.

A skilled person would understand the other conditions necessary to carry out the method according to any aspect of the present invention. In particular, the conditions in the container (e.g. fermenter) may be varied depending on the first, second and third microorganisms used. The varying of the conditions to be suitable for the optimal functioning of the microorganisms is within the knowledge of a skilled person.

In one example, the method according to any aspect of the present invention may be carried out in an aqueous medium with a pH between 5 and 8, 5.5 and 7. The pressure may be between 1 and 10 bar.

The term “contacting”, as used herein, means bringing about direct contact between the cell according to any aspect of the present invention and the medium comprising the carbon source in step (a) and/or the direct contact between the third microorganism and the acetate and/or ethanol from step (a) in step (b). For example, the cell, and the medium comprising the carbon source may be in different compartments in step (a). In particular, the carbon source may be in a gaseous state and added to the medium comprising the cells according to any aspect of the present invention.

In particular, the aqueous medium may comprise the cells and a carbon source comprising CO and/or CO₂ for step (a) to be carried out. More in particular, the carbon source comprising CO and/or CO₂ is provided to the aqueous medium comprising the cells in a continuous gas flow. Even more in particular, the continuous gas flow comprises synthesis gas. These gases may be supplied for example using nozzles that open up into the aqueous medium, frits, membranes within the pipe supplying the gas into the aqueous medium and the like.

The overall efficiency, alcohol productivity and/or overall carbon capture of the method of the present invention may be dependent on the stoichiometry of the CO₂, CO, and Hz in the continuous gas flow. The continuous gas flows applied may be of composition CO₂ and Hz. In particular, in the continuous gas flow, concentration range of CO₂ may be about 10-50%, in particular 3% by weight and Hz would be within 44% to 84%, in particular, 64 to 66.04% by weight. In another example, the continuous gas flow can also comprise inert gases like N₂, up to a N₂ concentration of 50% by weight.

The term ‘about’ as used herein refers to a variation within 20 percent. In particular, the term “about” as used herein refers to +/−20%, more in particular, +/−10%, even more in particular, +/−5% of a given measurement or value.

A skilled person would understand that it may be necessary to monitor the composition and flow rates of the streams. Control of the composition of the stream can be achieved by varying the proportions of the constituent streams to achieve a target or desirable composition. The composition and flow rate of the stream can be monitored by any means known in the art. In one example, the system is adapted to continuously monitor the flow rates and compositions of the streams and combine them to produce a single blended substrate stream in a continuous gas flow of optimal composition, and means for passing the optimised substrate stream to the cell according to any aspect of the present invention.

Microorganisms which convert CO₂ and/or CO to acetate and/or ethanol, in particular acetate, as well as appropriate procedures and process conditions for carrying out this metabolic reaction is well known in the art. Such processes are, for example described in WO9800558, WO2000014052 and WO2010115054.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep inventive cells, for example LB medium in the case of E. coli, ATCC1754-Medium may be used in the case of C. ljungdahlii. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium. The cells are incubated with the carbon source sufficiently long enough to produce the desired product, 3HB and variants thereof. For example for at least 1, 2, 4, 5, 10 or 20 hours. The temperature chosen must be such that the cells according to any aspect of the present invention remains catalytically competent and/or metabolically active, for example 10 to 42° C., preferably 30 to 40° C., in particular, 32 to 38° C. in case the cell is a C. ljungdahlii cell.

The expression “oxidation of an organic substance” according to any aspect of the present invention refers to for example, a hydroxylation or epoxidation, the reaction of an alkane to form an alcohol, the reaction of an alcohol to form an aldehyde or ketone, the reaction of an aldehyde to form a carboxylic acid, the reaction of an acid to form a rhamnolipid or the hydration of a double bond. Likewise, multistage oxidation processes are also summarized thereunder, as can be achieved, in particular, by using a plurality of oxidizing enzymes, such as, for example, the hydroxylation of an alkyl radical at a plurality of sites, e.g. at the 0) position and ω-1 position, catalysed by various monooxygenases.

The organic substance may be selected from the group consisting of branched or unbranched, saturated or unsaturated, optionally substituted alkanes, alkenes, alkynes, alcohols, aldehydes, ketones, carboxylic acids, esters of carboxylic acids, amines and epoxides. In particular, the organic substance may comprise 3 to 22, in particular 6 to 18, more in particular 8 to 14, even more in particular 12, carbon atoms.

In particular the organic substance that may be oxidised according to any aspect of the present invention may be selected from the group consisting of,

carboxylic acids and corresponding esters thereof, in particular having 3 to 22, more in particular 6 to 18, even more in particular 8 to 14 carbon atoms, in particular carboxylic acids of alkanes, in particular unbranched carboxylic acids of alkanes, in particular lauric acid and esters thereof, in particular lauric acid, methyl ester and lauric acid, ethyl ester, decanoic acid, esters of decanoic acid, myristic acid and esters of myristic acid, hexanoic acid and esters of hexanoic acid, octanoic acid and esters of octanoic acid and the like,

unsubstituted alkanes having 3 to 22, preferably 6 to 18, particularly preferably 8 to 14, carbon atoms, preferably unbranched, in particular selected from the group containing, preferably consisting of, octane, decane, dodecane and tetradecane,

unsubstituted alkenes having 3 to 22, preferably 6 to 18, particularly preferably 8 to 14, carbon atoms, preferably unbranched, in particular selected from the group containing, preferably consisting of, trans-oct-1-ene, trans-non-1-ene, trans-dec-1-ene, trans-undec-1-ene, trans-dodec-1-ene, trans-tridec-1-ene, trans-tetradec-1-ene, cis-oct-1-ene, cis-non-1-ene, cis-dec-1-ene, cis-undec-1-ene, cis-dodec-1-ene, cis-tridec-1-ene, cis-tetradec-1-ene, trans-oct-2-ene, trans-non-2-ene, trans-dec-2-ene, trans-undec-2-ene, trans-dodec-2-ene, trans-tridec-2-ene and trans-tetradec-2-ene, trans-oct-3-ene, trans-non-3-ene, trans-dec-3-ene, trans-undec-3-ene, trans-dodec-3-ene, trans-tridec-3-ene and trans-tetradec-3-ene, trans-oct-4-ene, trans-non-4-ene, trans-dec-4-ene, trans-undec-4-ene, trans-dodec-4-ene, trans-tridec-4-ene, trans-tetradec-4-ene, trans-dec-5-ene, trans-undec-5-ene, trans-dodec-5-ene, trans-tridec-5-ene, trans-tetradec-5-ene, trans-dodec-6-ene, trans-tridec-6-ene, trans-tetradec-6-ene, and trans-tetradec-7-ene, particularly preferably consisting of trans-oct-1-ene, trans-dec-1-ene, trans-dodec-1-ene, trans-tetradec-1-ene, cis-oct-1-ene, cis-dec-1-ene, cis-dodec-1-ene, cis-tetradec-1-ene, trans-oct-2-ene, trans-dec-2-ene, trans-dodec-2-ene and trans-tetradec-2-ene, trans-oct-3-ene, trans-dec-3-ene, trans-dodec-3-ene, and trans-tetradec-3-ene, trans-oct-4-ene, trans-dec-4-ene, trans-dodec-4-ene, trans-tetradec-4-ene, trans-dec-5-ene, trans-dodec-5-ene, trans-tetradec-5-ene, trans-dodec-6-ene, trans-tetradec-6-ene and trans-tetradec-7-ene,

unsubstituted monohydric alcohols having 3 to 22, preferably 6 to 18, particularly preferably 8 to 14, carbon atoms, preferably unbranched, in particular selected from the group containing, preferably consisting of, 1-butanol, 1-octanol, 1-nonanol, 1-decanol, 1-undecanol, 1-dodecanol, 1-tridecanol and 1-tetradecanol,

particularly preferably consisting of 1-octanol, 1-decanol, 1-dodecanol and 1-tetradecanol,

unsubstituted aldehydes having 3 to 22, preferably 6 to 18, particularly preferably 8 to 14, carbon atoms, preferably unbranched, in particular selected from the group containing, preferably consisting of, octanel, nonanal, decanal, dodecanal and tetradecanal,

unsubstituted monobasic amines having 3 to 22, preferably 6 to 18, particularly preferably 8 to 14, carbon atoms, preferably unbranched, in particular selected from the group containing, preferably consisting of, 1-aminooctane, 1-aminononane, 1-aminodecane, 1-aminoundecane, 1-aminododecane, 1-aminotridecane and 1-aminotetradecane,

particularly preferably consisting of 1-aminooctane, 1-aminodecane, 1-aminododecane and 1-aminotetradecane,

and also substituted compounds that, in particular, as further substituents, carry one or more hydroxyl, amino, keto, carboxyl, cyclopropyl radicals or epoxy functions, in particular selected from the group consisting of, 1,8-octanediol, 1,9-nonanediol, 1,10-decanediol, 1,11-undecanediol, 1,12-dodecanediol, 1,13-tridecanediol, 1,14-tetradecanediol, 8-amino-[1-octanol], 9-amino-[1-nonanol], 10-amino-[1-dodecanol], 11-amino-[1-undecanol], 12-amino-[1-dodecanol], 13-amino-[1-tridecanol], 14-amino-[1-tetradecanol], 8-hydroxy-[1-octanal], 9-hydroxy-[1-nonanal], 10-hydroxy-[1-decanal], 11-hydroxy-[1-undecanal], 12-hydroxy-[1-dodecanal], 13-hydroxy-[1-tridecanal], 14-hydroxy-[1-tetradecanal], 8-amino-[1-octanal], 9-amino-[1-nonanal], 10-amino-[1-decanal], 11-amino-[1-undecanal], 12-amino-[1-dodecanal], 13-amino-[1-tridecanal], 14-amino-[1-tetradecanal], 8-hydroxy-1-octanoic acid, 9-hydroxy-1-nonanoic acid, 10-hydroxy-1-decanoic acid, 11-hydroxy-1-undecanoic acid, 12-hydroxy-1-dodecanoic acid, 13-hydroxy-1-undecanoic acid, 14-hydroxy-1-tetradecanoic acid, 8-hydroxy-1-octanoic acid, methyl ester, 9-hydroxy-1-nonanoic acid, methyl ester, 10-hydroxy-1-decanoic acid, methyl ester, 11-hydroxy-1-undecanoic acid, methyl ester, 12-hydroxy-1-dodecanoic acid, methyl ester, 13-hydroxy-1-undecanoic acid, methyl ester, 14-hydroxy-1-tetradecanoic acid, methyl ester, 8-hydroxy-1-octanoic acid, ethyl ester, 9-hydroxy-1-nonanoic acid, ethyl ester, 10-hydroxy-1-decanoic acid, ethyl ester, 11-hydroxy-1-undecanoic acid, ethyl ester, 12-hydroxy-1-dodecanoic acid, ethyl ester, 13-hydroxy-1-undecanoic acid, ethyl ester and 14-hydroxy-1-tetra-decanoic acid, ethyl ester,

particularly preferably consisting of 1,8-octanediol, 1,10-decanediol, 1,12-dodecanediol, 1,14-tetradecanediol, 8-amino-[1-octanol], 10-amino-[1-dodecanol], 12-amino-[1-dodecanol], 14-amino-[1-tetradecanol], 8-hydroxy-[1-octanal], 10-hydroxy-[1-decanal], 12-hydroxy-[1-dodecanal], 14-hydroxy-[1-tetradecanal], 8-amino-[1-octanal], 10-amino-[1-decanal], 12-amino-[1-dodecanal], 14-amino-[1-tetradecanal], 8-hydroxy-1-octanoic acid, 10-hydroxy-1-decanoic acid, 12-hydroxy-1-dodecanoic acid, 14-hydroxy-1-tetradecanoic acid, 8-hydroxy-1-octanoic acid, methyl ester, 10-hydroxy-1-decanoic acid, methyl ester, 12-hydroxy-1-dodecanoic acid, methyl ester, 14-hydroxy-1-tetradecanoic acid, methyl ester, 8-hydroxy-1-octanoic acid, ethyl ester, 10-hydroxy-1-decanoic acid, ethyl ester, 12-hydroxy-1-dodecanoic acid, ethyl ester and 14-hydroxy-1-tetradecanoic acid, ethyl ester,

wherein in particular lauric acid and esters thereof, more in particular lauric acid, methyl ester and lauric acid, ethyl ester, may be used.

By means of the method according to any aspect of the present invention, depending on the oxidizing enzyme used and the organic substance used, various oxidation products may be produced, in particular alcohols, aldehydes, ketones and carboxylic acids. These oxidation products may be obtained, for example, by means of the method according to any aspect of the present invention by reacting an organic substance listed herein to form the following:
alkane/alkene/alkyne to form alcohol (for example in the presence of a monooxygenase)
alcohol to form aldehyde (for example in the presence of an alcohol dehydrogenase or alcohol oxidase)
alcohol to form ketone (for example in the presence of an alcohol dehydrogenase or alcohol oxidase)
alcohol to form carboxylic acid (for example in the presence of an alcohol dehydrogenase)
aldehyde to form carboxylic acid (for example in the presence of an aldehyde dehydrogenase)
epoxide to form cyanohydrin (for example in the presence of a halohydrin dehalogenase)
a pyruvate to form acetate (for example in the presence of pyruvate decarboxylase)
a carboxylic acid to form alkene (for example in the presence of carboxylic acid reductase) or a rhamnolipid (for example in the presence of an α/β hydrolase (RH/A), rhamnosyltransferase I (RNIB) and a rhamnosyltransferase II (RHIC).

Within this context, preference is given to producing alcohols and aldehydes, preferably alcohols, in particular ω-alcohols, very particularly ω-hydroxycarboxylic acids using the method according to the invention, in particular in the form of a hydroxylation reaction. In one example, butyric acid is produced from butanol used as the organic substance according to any aspect of the present invention.

In the method according to the invention, all oxidizing enzymes known to those skilled in the art may be used. Such enzymes are well known to those skilled in the art under the name oxidoreductase and may be found in enzyme class EC 1.X.X.X of the systematic nomenclature of the Enzyme Commission of the International Union of Biochemistry and Molecular Biology. In particular, the oxidising enzyme may be selected from the group consisting of alkane monooxygenase, a xylene monooxygenase, an aldehyde dehydrogenase, an alcohol oxidase and an alcohol dehydrogenase. In particular, the oxidising enzyme may be alkane monooxygenase.

A suitable gene for a xylene monooxygenase may be, for example, the xylM or the xylA gene, wherein a plasmid containing these two genes has the GENBANK Accession No. M37480.

A particularly preferred alkane monooxygenase within this context may be characterized in that it is a cytochrome-P450 monooxygenase, in particular a cytochrome-P450 monooxygenase from yeasts, in particular Pichia, Yarrowia and Candida, for example from Candida tropicalis or Candida maltose, or from plants, for example from Cicer arietinum L., or from mammals, for example from Rattus norvegicus, in particular CYP4A1. The gene sequences of suitable cytochrome-P450 monooxygenases from Candida tropicalis are disclosed, for example, in WO-A-00/20566, while the gene sequences of suitable cytochrome-P450 monooxygenases from chickpea may be found, for example, in Barz et al., 2000.

A further preferred alkane monooxygenase may be encoded by the alkB gene of the alk operon from Pseudomonas putida GPo1. The isolation of the alkB gene sequence is described, for example, by van Beilen et al., 2002. Further homologues of the alkB gene can also be found from van Beilen et al. 2003. In addition, preferred alkane monooxygenases are those a/kB gene products which are encoded by a/kB genes from organisms selected from the group of the Gram-negative bacteria, in particular from the group of the Pseudomonads, there from the genus Pseudomonas, particularly Pseudomonas mendocina, the genus Oceanicaulis, preferably Oceanicaulis alexandrii HTCC2633, the genus Caulobacter, preferably Caulobacter sp. K31, the genus Marinobacter, preferably Marinobacter aquaeolei, particularly preferably Marinobacter aquaeolei VT8, the genus Alcanivorax, preferably Alcanivorax borkumensis, the genus Acetobacter, Achromobacter, Acidiphilium, Acidovorax, Aeromicrobium, Alkalilimnicola, Alteromonadales, Anabaena, Aromatoleum, Azoarcus, Azospirillum, Azotobacter, Bordetella, Bradyrhizobium, Burkholderia, Chlorobium, Citreicella, Clostridium, Colwellia, Comamonas, Conexibacter, Congregibacter, Corynebacterium, Cupriavidus, Cyanothece, Delftia, Desulfomicrobium, Desulfonatronospira, Dethiobacter, Dinoroseobacter, Erythrobacter, Francisella, Glaciecola, Gordonia, Grimontia, Hahella, Haloterrigena, Halothiobacillus, Hoeflea, Hyphomonas, Janibacter, Jannaschia, Jonquetella, Klebsiella, Legionella, Limnobacter, Lutiella, Magnetospirillum, Mesorhizobium, Methylibium, Methylobacterium, Methylophaga, Mycobacterium, Neisseria, Nitrosomonas, Nocardia, Nostoc, Novosphingobium, Octadecabacter, Paracoccus, Parvibaculum, Parvularcula, Peptostreptococcus, Phaeobacter, Phenylobacterium, Photobacterium, Polaromonas, Prevotella, Pseudoalteromonas, Pseudovibrio, Psychrobacter, Psychroflexus, Ralstonia, Rhodobacter, Rhodococcus, Rhodoferax, Rhodomicrobium, Rhodopseudomonas, Rhodospirillum, Roseobacter, Roseovarius, Ruegeria, Sagittula, Shewanella, Silicibacter, Stenotrophomonas, Stigmatella, Streptomyces, Sulfitobacter, Sulfurimonas, Sulfurovum, Synechococcus, Thalassiobium, Thermococcus, Thermomonospora, Thioalkalivibrio, Thiobacillus, Thiomicrospira, Thiomonas, Tsukamurella, Vibrio or Xanthomonas, wherein those from Alcanivorax borkumensis, Oceanicaulis alexandrii HTCC2633, Caulobacter sp. K31 and Marinobacter aquaeolei VT8 are particularly preferred. In this context, it is advantageous if, in addition to AlkB, alkG and alkT gene products are provided; these can either be the gene products isolatable from the organism contributing the alkB gene product, or else the alkG and alkT from Pseudomonas putida GPo1.

A preferred alcohol may be for example, the enzyme (EC 1.1.99.8) encoded by the a/kJ gene, in particular the enzyme encoded by the a/kJ gene from Pseudomonas putida GPo1 (van Beilen et al, 1992). The gene sequences of the a/kJ genes from Pseudomonas putida GPo1, Alcanivorax borkumensis, Bordetella parapertussis, Bordetella bronchiseptica or from Roseobacter denitrificans can be found, for example, in the KEGG gene database (Kyoto Encylopedia of Genes and Genomes). In addition, preferred alcohol dehydrogenases are those which are encoded by a/kJ genes from organisms selected from the group of the Gram-negative bacteria, in particular from the group of the Pseudomonads, there from the genus Pseudomonas, particularly Pseudomonas mendocina, the genus Oceanicaulis, preferably Oceanicaulis alexandrii HTCC2633, the genus Caulobacter, preferably Caulobacter sp. K31, the genus Marinobacter, preferably Marinobacter aquaeolei, particularly preferably Marinobacter aquaeolei VT8, the genus Alcanivorax, preferably Alcanivorax borkumensis, the genus Acetobacter, Achromobacter, Acidiphilium, Acidovorax, Aeromicrobium, Alkalilimnicola, Alteromonadales, Anabaena, Aromatoleum, Azoarcus, Azospirillum, Azotobacter, Bordetella, Bradyrhizobium, Burkholderia, Chlorobium, Citreicella, Clostridium, Colwellia, Comamonas, Conexibacter, Congregibacter, Corynebacterium, Cupriavidus, Cyanothece, Delftia, Desulfomicrobium, Desulfonatronospira, Dethiobacter, Dinoroseobacter, Erythrobacter, Francisella, Glaciecola, Gordonia, Grimontia, Hahella, Haloterrigena, Halothiobacillus, Hoeflea, Hyphomonas, Janibacter, Jannaschia, Jonquetella, Klebsiella, Legionella, Limnobacter, Lutiella, Magnetospirillum, Mesorhizobium, Methylibium, Methylobacterium, Methylophaga, Mycobacterium, Neisseria, Nitrosomonas, Nocardia, Nostoc, Novosphingobium, Octadecabacter, Paracoccus, Parvibaculum, Parvularcula, Peptostreptococcus, Phaeobacter, Phenylobacterium, Photobacterium, Polaromonas, Prevotella, Pseudoalteromonas, Pseudovibrio, Psychrobacter, Psychroflexus, Ralstonia, Rhodobacter, Rhodococcus, Rhodoferax, Rhodomicrobium, Rhodopseudomonas, Rhodospirillum, Roseobacter, Roseovarius, Ruegeria, Sagittula, Shewanella, Silicibacter, Stenotrophomonas, Stigmatella, Streptomyces, Sulfitobacter, Sulfurimonas, Sulfurovum, Synechococcus, Thalassiobium, Thermococcus, Thermomonospora, Thioalkalivibrio, Thiobacillus, Thiomicrospira, Thiomonas, Tsukamurella, Vibrio or Xanthomonas.

Preferred alkL gene products used in the method according to any aspect of the present invention are characterized in that the production of the alkL gene product is induced in the native host by dicyclopropyl ketone; in this context it is, in addition, preferred that the alkL gene is expressed as part of a group of genes, for example in a regulon, such as, for instance, an operon. The alkL gene products used in the method according to any aspect of the present invention are preferably encoded by alkL genes from organisms selected from the group of the Gram-negative bacteria, in particular the group containing, preferably consisting of, Pseudomonads, particularly Pseudomonas putida, in particular Pseudomonas putida GPo1 and P1, Azotobacter, Desulfitobacterium, Burkholderia, preferably Burkholderia cepacia, Xanthomonas, Rhodobacter, Ralstonia, Delftia and Rickettsia, the genus Oceanicaulis, preferably Oceanicaulis alexandrii HTCC2633, the genus Caulobacter, preferably Caulobacter sp. K31, the genus Marinobacter, preferably Marinobacter aquaeolei, particularly preferably Marinobacter aquaeolei VT8 and the genus Rhodopseudomonas. It is advantageous if the alkL gene product originates from a different organism from the oxidizing enzyme used according to the invention. In this context, very particularly preferred alkL gene products are encoded by the alkL genes from Pseudomonas putida GPo1 and P1, which are given by SEQ ID NO:1 and SEQ ID NO:3, and also proteins having the polypeptide sequence SEQ ID NO:2 or SEQ ID NO:4 or having a polypeptide sequence in which up to 60%, preferably up to 25%, particularly preferably up to 15%, in particular up to 10, 9, 8, 7, 6, 5, 4, 3, 2, 1% of the amino acid residues are modified in comparison with SEQ ID NO:2 or SEQ ID NO:4 by deletion, insertion, substitution or a combination thereof and which products still have at least 50%, preferably 65%, particularly preferably 80%, in particular more than 90%, of the activity of the protein having the respective reference sequence SEQ ID NO:2 or SEQ ID NO:4, wherein 100% activity of the reference protein is taken to mean the increase of the activity of the cells used as biocatalyst, that is to say the amount of substance reacted per unit time, based on the cell weight used (units per gram of cell dry weight [U/gCDW]), compared with the activity of the biocatalyst without the presence of the reference protein, more precisely in a system as described in the exemplary embodiments, in which the oxidizing enzymes used for converting lauric acid, methyl ester to 12-hydroxylauric acid, methyl ester in an E. coli cell are the gene products of alkBGT from P. putida GPo1.

The definition of the unit here is the definition customary in enzyme kinetics. One unit of biocatalyst reacts 1 μmol of substrate in 1 minute to form the product.

1 U=1 μmol/min

Modifications of amino acid residues of a given polypeptide sequence that do not lead to any substantial changes of the properties and function of the given polypeptide are known to those skilled in the art. For instance, some amino acids, for example, can frequently be exchanged for one another without problem; examples of such suitable amino acid substitutions are: Ala for Ser; Arg for Lys; Asn for Gln or His; Asp for Glu; Cys for Ser; Gln for Asn; Glu for Asp; Gly for Pro; His for Asn or Gln; Ile for Leu or Val; Leu for Met or Val; Lys for Arg or Gln or Glu; Met for Leu or Ile; Phe for Met or Leu or Tyr; Ser for Thr; Thr for Ser; Trp for Tyr; Tyr for Trp or Phe; Val for Ile or Leu. It is likewise known that modifications particularly at the N- or C-terminus of a polypeptide in the form of, for example, amino acid insertions or deletions frequently have no substantial effect on the function of the polypeptide.

In particular, according to any aspect of the present invention,
(a) the alkane monooxygenase may be a cytochrome-P450 monooxygenase;
(b) the alkane monooxygenase may be an alkB gene product which is encoded by an alkB gene from at least one Gram-negative bacteria; and/or
(c) the alcohol dehydrogenase may be the alcohol dehydrogenase encoded by the alkJ gene from at least one Gram-negative bacteria.

More in particular, the Gram-negative bacteria may be selected from the group consisting of Pseudomonads, Azotobacter, Desulfitobacterium, Burkholderia, Xanthomonas, Rhodobacter, Ralstonia, Deiftia, Rickettsia, Oceanicaulis, Caulobacter, Marinobacter, and Rhodopseudomonas. In particular, the alkL gene product comprises an amino acid sequence selected from the group consisting of SEQ ID Nos: 1-4.

In particular, according to any aspect of the present invention, the third microorganism may be genetically modified to increase expression of at least one oxidising enzyme relative to the wild type cell, wherein the oxidising enzyme is selected from the group consisting of alkane monooxygenase, a xylene monooxygenase, an aldehyde dehydrogenase, an alcohol oxidase and an alcohol dehydrogenase.

In one example, the the first and/or second microorganism is Clostridium ljungdahlii and the third microorganism is Escherichia coli.

Step (a) and step (b) may be carried out in two different containers. In one example, step (a) may be carried out in fermenter 1 wherein the first and second microorganisms come in contact with the carbon source to produce acetate and/or ethanol. Ethanol and/or acetate may then be brought into contact with a third microorganism in fermenter 2 to produce at least one amino acid. The amino acid and/or the desired amino acid may then be extracted and the remaining carbon substrate fed back into fermenter 1. A cycle may be created wherein the acetate and/or ethanol produced in fermenter 1 may be regularly fed into fermenter 2, the acetate and/or ethanol in fermenter 2 may be converted to at least one amino acid and the unreacted carbon source in fermenter 2 fed back into fermenter 1. Oxygen may be added into fermenter 2 to enable the third microorganism to convert acetate to at least one amino acid. When the remaining carbon source is cycled back from fermenter 2 to fermenter 1, consequently small amounts of oxygen and amino acids may enter fermenter 1. The presence of these small amounts of oxygen and amino acids may still allow for the first and second microorganisms to carry out their activity of converting carbon to acetate and/or ethanol.

In another example, the media is being recycled between fermenters 1 and 2. Therefore, the amino acid produced in fermenter 2 may be fed back into fermenter 1 to accumulate the amino acid produced according to any aspect of the present invention in the fermenters. In the process of recycling the media, oxygen from fermenter 2 and the amino acids produced in fermenter 2 are consequently reintroduced into fermenter 1. As can be seen in the examples, the amino acids may not be metabolised by the microorganisms in fermenter 1. Accordingly, the amino acids may accumulate in the media within the two fermenters. Also, the microorganisms in fermenter 1 may be able to continue producing acetate and ethanol in the presence of the oxygen recycled from fermenter 2 into fermenter 1. The accumulated amino acids may then be extract by means known in the art.

Means of extracting amino acids according to any aspect of the present invention may include an aqueous two-phase system for example comprising polyethylene glycol, capillary electrolysis, chromatography and the like. In one example, when chromatography is used as the means of extraction, ion exchange columns may be used. In another example, amino acids may be precipitated using pH shifts. A skilled person may easily identify the most suitable means of extracting amino acids by simple trial and error.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Oxidation of Butanol to Butyric Acid with Escherichia coli and Glucose as Co-Substrate

For the biotransformation of butanol to butyrate the plasmid harboring strain E. coli W3110 ΔfadE pBT10 was used. The plasmid pBT10 is described in WO2009/077461 and the E. coli strain is described in WO2013/092547.

The recombinant E. coli W3110 ΔfadE pBT10 was cultivated on plate count agar (Merck, Germany) with 50 mg/l kanamycin.

For a first preculture 25 mL of LB medium (Merck, Germany) with 50 mg/L kanamycin in a 250 mL shaking flask were inoculated with a single colony from a fresh incubated agar plate and cultivated at 37° C. and 200 rpm for 16 h. For a second preculture 100 mL of HZD medium (1.8 g/L (NH₄)₂SO₄, 19.1 g/L K₂HPO₄, 12.5 g/L KH₂PO₄, 6.7 g/L yeast extract, 2.3 g/L Na₃-Citrat*2H₂O, 170 mg/L NH₄Fe-Citrat, 5 mL/L trace elements US3 (80 mL/L 37% HCl, 1.9 g/L MnCl₂*4H₂O, 1.9 g/L ZnSO₄*7H₂O, 0.9 g/L Na-EDTA*2H₂O, 0.3 g/l H₃BO₃, 0.3 g/L Na₂MoO₄*2H₂O, 4.7 g/L CaCl₂*2H₂O, 17.8 g/L FeSO₄*7H₂O, 0.2 g/L CuCl₂*2H₂O), 30 mL/L HZD-feed (50 g/kg Glucose×H₂O, 10 g/kg MgSO₄×7H₂O, 22 g/kg NH₄Cl)) with 50 mg/L kanamycin in a 1000 mL shaking flask were inoculated with OD_{600 nm} of 0.1 from the first preculture and cultivated at 37° C. and 200 rpm for 8 h. For the main culture 15 L of fresh HZD medium (pH 6.8) with 15 g/L glucose in a 20 L stirred tank bioreactor were inoculated with cells from the second preculture to an OD_{600 nm} of 0.1. The fermentation was carried out at 37° C. and 30% pO₂ (250 to 1200 rpm with an airflow of 4.2-30 L/min). The pH was maintained at 6.8 with 25% NH₄OH. When the pO₂ reached 45% a feed with 5 g/L*h glucose was started. 4 h before harvest the culture was induced with 0.025% DCPK. After harvest at high OD_{600 nm} the cells were centrifuged and stored at −20° C.

For the oxidation reaction 15 mL assay buffer (pH 7.4, 1.347 g/L KH₂PO₄, 6.98 g/L K₂HPO₄, 0.5 g/L NH₄Cl) with 1 g/L butanol as substrate and 1 g/l glucose as co-substrate in a 50 mL reaction tube were inoculated with 1.6 g/L washed cells from the frozen stock of the main culture and incubated at 30° C. and 300 rpm in a water bath shaker for 30 h.

At the start and during the incubation period, samples were taken. These were tested for optical density, pH and the different analytes. The determination of the product concentrations was performed by semi quantitative ¹H-NMR spectroscopy. As an internal quantification standard sodium trimethylsilylpropionate (T(M)SP) was used.

During the incubation period of 30 h the concentration of glucose decreased from 847 mg/L to 0 mg/L, the concentration of butanol decreased from 1046 to 277 mg/L and the concentration of butyrate increased from 0 to 973 g/L.

### Example 2

Oxidation of Butanol to Butyric Acid with Escherichia coli and Acetate as Co-Substrate

For the biotransformation of butanol to butyrate the plasmid harboring strain E. coli W3110 ΔfadE pBT10 was used. The plasmid pBT10 is described in WO2009/077461 and the E. coli strain is described in WO2013/092547.

The recombinant E. coli W3110 ΔfadE pBT10 was cultivated on plate count agar (Merck, Germany) with 50 mg/l kanamycin.

For a first preculture 25 mL of LB medium (Merck, Germany) with 50 mg/L kanamycin in a 250 mL shaking flask were inoculated with a single colony from a fresh incubated agar plate and cultivated at 37° C. and 200 rpm for 16 h. For a second preculture 100 mL of HZD medium (1.8 g/L (NH₄)₂SO₄, 19.1 g/L K₂HPO₄, 12.5 g/L KH₂PO₄, 6.7 g/L yeast extract, 2.3 g/L Na₃-Citrate*2H₂O, 170 mg/L NH₄Fe-Citrate, 5 mL/L trace elements US3 (80 mL/L 37% HCl, 1.9 g/L MnCl₂*4H₂O, 1.9 g/L ZnSO₄*7H₂O, 0.9 g/L Na-EDTA*2H₂O, 0.3 g/l H₃BO₃, 0.3 g/L Na₂MoO₄*2H₂O, 4.7 g/L CaCl₂*2H₂O, 17.8 g/L FeSO₄*7H₂O, 0.2 g/L CuCl₂*2H₂O), 30 mL/L HZD-feed (50 g/kg Glucose×H₂O, 10 g/kg MgSO₄×7H₂O, 22 g/kg NH₄Cl)) with 50 mg/L kanamycin in a 1000 mL shaking flask were inoculated with OD_{600 nm} of 0.1 from the first preculture and cultivated at 37° C. and 200 rpm for 8 h.

For the main culture 15 L of fresh HZD medium (pH 6.8) with 15 g/L glucose in a 20 L stirred tank bioreactor were inoculated with cells from the second preculture to an OD_{600 nm} of 0.1. The fermentation was carried out at 37° C. and 30% pO₂ (250 to 1200 rpm with an airflow of 4.2-30 L/min). The pH was hold at 6.8 with 25% NH₄OH. When the pO₂ reached 45% a feed with 5 g/L*h glucose was started. 4 h before harvest the culture was induced with 0.025% DCPK. After harvest at high OD_{600 nm} the cells were centrifuged and stored at −20° C.

For the oxidation reaction 15 mL assay buffer (pH 7.4, 1.347 g/L KH₂PO₄, 6.98 g/L K₂HPO₄, 0.5 g/L NH₄Cl) with 1 g/L butanol as substrate and 1.72 g/l potassium acetate as co-substrate in a 50 mL reaction tube were inoculated with 1.6 g/L washed cells from the frozen stock of the main culture and incubated at 30° C. and 300 rpm in a water bath shaker for 30 h.

At the start and during the incubation period, samples were taken. These were tested for optical density, pH and the different analytes. The determination of the product concentrations was performed by semi quantitative ¹H-NMR spectroscopy. As an internal quantification standard sodium trimethylsilylpropionate (T(M)SP) was used.

During the incubation period of 30 h the concentration of acetate decreased from 1683 mg/L to 0 mg/L, the concentration of butanol decreased from 1023 to 0 mg/L and the concentration of butyrate increased from 0 to 1216 g/L.

### Example 3

Production of Acetate and Ethanol with Clostridium ljungdahlii from Synthesis Gas without Oxygen

In this example, C. ljungdahlii was anaerobically cultivated in complex medium with synthesis gas, consisting of Hz and CO₂ in the absence of oxygen in order to produce acetate and ethanol. For cell culture of C. ljungdahlii 2 mL Cryoculture was cultured anaerobically in 200 ml of medium (ATCC1754 medium: pH 6.0; 20 g/L MES; 1 g/L yeast extract, 0.8 g/L NaCl, 1 g/L NH₄Cl, 0.1 g/L KCl, 0.1 g/L KH₂PO₄, 0.2 g/L MgSO₄×7H₂O; 0.02 g/L CaCl₂×2H₂O; 20 mg/L nitrilotriacetic acid 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B12; 50 μg/L p-aminobenzoate; 50 μg/L lipoic acid, approximately 67.5 mg/L NaOH) with about 400 mg/L L-cysteine hydrochloride and 400 mg/L Na₂S×9H₂O. Cultivation was carried chemolithoautotrophically in a flameproof 1 L glass bottle with a premixed gas mixture composed of 67% Hz, 33% CO₂ in an open water bath shaker at 37° C., 150 rpm and a fumigation of 1-3 L/h for 161 h. The gas entry into the medium was carried out by a filter with a pore size of 10 microns, and was mounted in the middle of the reactor, at a gassing tube. The cells were centrifuged, washed with 10 ml ATCC medium and centrifuged again.

For the preculture many washed cells from the growth culture of C. ljungdahlii were transferred into 200 mL of ATCC medium with about 400 mg/L L-cysteine hydrochloride and grown to an OD₆₀₀ of 0.12. Cultivation was carried out in a pressure-resistant 500 ml glass bottle with a premixed gas mixture composed of 67% Hz, 33% CO₂, in an open water bath shaker at 37° C., 150 rpm and with aeration of 3 L/h for 65 h. The gas entry into the medium was carried out by a filter with a pore size of 10 microns, which was placed in the middle of the reactors. The cells were centrifuged, washed with 10 ml of production buffer (pH 6.2; 0.5 g/L of KOH, aerated for 1 h with a premixed gas mixture of 67% Hz, 33% CO₂ at 1 L/hr) washed and centrifuged again.

For the production culture many of washed cells from the preculture of C. ljungdahlii were transferred into 200 mL of ATCC medium with about 400 mg/L L-cysteine hydrochloride and grown to an OD₆₀₀ of 0.2. Cultivation was carried out in a pressure-resistant 500 ml glass bottle with a premixed gas mixture composed of 67% Hz, 33% CO₂, in an open water bath shaker at 37° C., 150 rpm and with aeration of 3 L/h for 118 h. The gas entry into the medium was carried out by a filter with a pore size of 10 microns, which was placed in the middle of the reactors. When the pH fell below 5.0, 1 ml of a 140 g/l KOH solution was added. When sampling each 5 ml sample was removed for determination of OD₆₀₀, pH and the product range. The determination of the product concentration was performed by semi-quantitative 1H-NMR spectroscopy. As an internal quantification standard sodium trimethylsilylpropionate served (T(M)SP).

Over the culturing period of 118 h, the cell density in the production culture remained constant, recognizable by a stagnant OD₆₀₀ of 0.2, corresponding to a growth rate of p=0 hr⁻¹. The concentration of acetate increased significantly at the same time from 4 mg/L to 3194 mg/L and the concentration of ethanol from 17 mg/L to 108 mg/L.

### Example 4

No Production of Acetate and Ethanol with Clostridium ljungdahlii from Synthesis Gas Comprising CO₂ and H₂ with Oxygen

C. ljungdahlii was cultivated in complex medium with synthesis gas and oxygen. C. ljungdahlii was first cultured in the presence of synthesis gas consisting of Hz and CO₂ in the absence of oxygen in order to produce acetate and ethanol. For the cultivation, the cells were grown in pressure-resistant glass bottles that could be sealed airtight with a butyl rubber stopper. All steps in which C. ljungdahlii cells were involved were carried out under anaerobic conditions.

For cell culture of C. ljungdahlii 2 mL Cryoculture was cultured anaerobically in 200 ml of medium (ATCC1754 medium: pH 6.0; 20 g/L MES; 1 g/L yeast extract, 0.8 g/L NaCl, 1 g/L NH₄Cl, 0.1 g/L KCl, 0.1 g/L KH₂PO₄, 0.2 g/L MgSO₄×7H₂O; 0.02 g/L CaCl₂×2H₂O; 20 mg/L nitrilotriacetic acid 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B12; 50 μg/L p-aminobenzoate; 50 μg/L lipoic acid, approximately 67.5 mg/L NaOH) with about 400 mg/L L-cysteine hydrochloride and 400 mg/L Na₂S×9H₂O. Cultivation was carried chemolithoautotrophically in a flameproof 1 L glass bottle with a premixed gas mixture composed of 67% Hz, 33% CO₂ in an open water bath shaker at 37° C., 150 rpm and a fumigation of 1-3 L/h for 161 h. The gas entry into the medium was carried out by a filter with a pore size of 10 microns, and was mounted in the middle of the reactor, at a gassing tube. The cells were centrifuged, washed with 10 ml ATCC medium and centrifuged again.

For the preculture many washed cells from the growth culture of C. ljungdahlii were transferred into 200 mL of ATCC medium with about 400 mg/L L-cysteine hydrochloride and grown to an OD₆₀₀ of 0.12. Cultivation was carried out in a pressure-resistant 500 ml glass bottle with a premixed gas mixture composed of 67% Hz, 33% CO₂, in an open water bath shaker at 37° C., 150 rpm and with aeration of 3 L/h for 24 h. Subsequently, the gas mixture was changed to one with the composition of 66.85% H₂, 33% CO₂ and 0.15% Oz and the cells were further gassed for 67 h at 3 L/h. The gas entry into the medium was carried out by a Begasungsfritte with a pore size of 10 microns, which was placed in the middle of the reactors at a sparger. The cells were centrifuged, washed with 10 ml ATCC medium and centrifuged again. The gas entry into the medium was carried out by a filter with a pore size of 10 microns, which was placed in the middle of the reactors. The cells were centrifuged, washed with 10 ml of ATCC medium and centrifuged again.

For the production culture many of washed cells from the preculture of C. ljungdahlii were transferred into 200 mL of ATCC medium with about 400 mg/L L-cysteine hydrochloride and grown to an OD₆₀₀ of 0.1. Cultivation was carried out in a pressure-resistant 500 ml glass bottle with a premixed gas mixture composed of 66.85% Hz, 33% CO₂ and 0.15% O₂, in an open water bath shaker at 37° C., 150 rpm and with aeration of 3 L/h for 113 h. The gas entry into the medium was carried out by a filter with a pore size of 10 microns, which was placed in the middle of the reactors. When sampling each 5 ml sample was removed for determination of OD₆₀₀, pH and the product range. The determination of the product concentration was performed by semi-quantitative 1H-NMR spectroscopy. As an internal quantification standard sodium trimethylsilylpropionate served (T (M) SP).

In the period from 89 h to 113 h there was no recognizable cell growth shown. The OD₆₀₀ was stagnated at 0.29, corresponding to a growth rate p=0 h⁻¹ The concentration of acetate increased slightly during this time from 89.4 mg/L to 86.9 mg/L and the concentration of ethanol decreased from 16.2 mg/L to 11.9 mg/L.

### Example 5

Culture of Clostridium ljungdahlii in Log Phase in the Presence of Synthesis Gas Comprising CO₂ and 0.15% Oxygen

C. ljungdahlii was fed Hz and CO₂ out of the feed-through gas phase and formed acetate and ethanol. For the cultivation, pressure-resistant glass bottle that can be sealed airtight with a butyl rubber stopper were used. All cultivation steps, where C. ljungdahlii cells were involved were carried out under anaerobic conditions.

For cell culture of C. ljungdahlii 5 mL Cryoculture was cultured anaerobically in 500 ml of medium (ATCC1754 medium: pH 6.0; 20 g/L MES; 1 g/L yeast extract, 0.8 g/L NaCl, 1 g/L NH₄Cl, 0.1 g/L KCl, 0.1 g/L KH₂PO₄, 0.2 g/L MgSO₄×7H₂O; 0.02 g/L CaCl₂×2H₂O; 20 mg/L nitrilotriacetic acid 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B12; 50 μg/L p-aminobenzoate; 50 μg/L lipoic acid, approximately 67.5 mg/L NaOH) with about 400 mg/L L-cysteine hydrochloride and 400 mg/L Na₂S×9H₂O. Cultivation was carried chemolithoautotrophically in a flameproof 1 L glass bottle with a premixed gas mixture composed of 67% Hz, 33% CO₂ in an open water bath shaker at 37° C., 100 rpm and a fumigation of 3 L/h for 72 h. The gas entry into the medium was carried out by a filter with a pore size of 10 microns, and was mounted in the middle of the reactor, at a gassing tube. The cells were centrifuged, washed with 10 ml ATCC medium and centrifuged again.

For the main culture many washed cells from the growth culture of C. ljungdahlii were transferred into 500 mL of ATCC medium with about 400 mg/L L-cysteine hydrochloride and grown to an OD₆₀₀ of 0.1. Cultivation was carried out in a pressure-resistant 1 L glass bottle with a premixed gas mixture composed of 66.85% H₂, 33% CO₂, 0.15% O₂ in an open water bath shaker at 37° C., 150 rpm and with aeration of 1 L/h for 45 h. The gas entry into the medium was carried out by a filter with a pore size of 10 microns, which was placed in the middle of the reactors. When sampling each 5 ml sample was removed for determination of OD_{600 nm}, pH and the product range. The determination of the product concentration was performed by semi-quantitative 1H-NMR spectroscopy. As an internal quantification standard sodium trimethylsilylpropionate served (T (M) SP).

There was significant cell growth shown during the cultivation period, evidenced by an increase in OD_{600 nm} of 0.10 to 0.54, corresponding to a growth rate p=0.037 h⁻¹. The concentration of acetate increased at the same time from 9.6 mg/L to 3,304 mg/L and the concentration of ethanol from 2.2 mg/L to 399 mg/L.

### Example 6

Culture of Clostridium ljungdahlii in Log Phase in the Presence of Synthesis Gas Comprising CO and 0.1% Oxygen

C. ljungdahlii was autotrophically cultivated in complex medium with synthesis gas, consisting of CO, Hz and CO₂ in the presence of oxygen in order to produce acetate and ethanol.

A complex medium was used consisting of 1 g/L NH₄Cl, 0.1 g/L KCl, 0.2 g/L MgSO₄×7H₂O, 0.8 g/L NaCl, 0.1 g/L KH₂PO₄, 20 mg/L CaCl₂×2H₂O, 20 g/L MES, 1 g/L yeast extract, 0.4 g/L L-cysteine-HCl, 0.4 g/L Na₂S×9H₂O, 20 mg/L nitrilotriacetic acid, 10 mg/L MnSO₄×H₂O, 8 mg/L (NH₄)₂Fe(SO₄)₂×6H₂O, 2 mg/L CoCl₂×6H₂O, 2 mg/L ZnSO₄×7H₂O, 0.2 mg/L CuCl₂×2H₂O, 0.2 mg/L Na₂MoO₄×2H₂O, 0.2 mg/L NiCl₂×6H₂O, 0.2 mg/L Na₂SeO₄, 0.2 mg/L Na₂WO₄×2H₂O, 20 μg/L biotin, 20 μg/L folic acid, 100 μg/L pyridoxine-HCl, 50 μg/L thiamine-HCl×H₂O, 50 μg/L riboflavin, 50 μg/L nicotinic acid, 50 μg/L Ca-pantothenoic acid, 1 μg/L vitamine B12, 50 μg/L p-aminobenzoic acid, 50 μg/L lipoic acid.

The autotrophic cultivation was performed in 500 mL medium in a 1 L serum bottle that was continuously gassed with synthesis gas consisting of 67.7% CO, 3.5% H₂ and 15.6% CO₂ at a rate of 3.6 L/h. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 120 min⁻¹. pH was not controlled.

At the beginning of the experiment, C. ljungdahlii was inoculated with an OD₆₀₀ of 0.1 with autotrophically grown cells on H₂/CO₂. Therefore, C. ljungdahlii was grown in complex medium under continuous gassing with synthesis gas consisting of 67% H₂ and 33% CO₂ at a rate of 3 L/h in 1 L serum bottles with 500 mL complex medium. Above described medium was also used for this cultivation. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 150 min⁻¹. The cells were harvested in the logarithmic phase with an OD₆₀₀ of 0.49 and a pH of 5.03 by anaerobic centrifugation (4500 min⁻¹, 4300 g, 20° C., 10 min). The supernatant was discarded and the pellet was resuspended in 10 mL of above described medium. This cell suspension was then used to inoculate the cultivation experiment. Gas phase concentration of carbon monoxide was measured sampling of the gas phase and offline analysis by an gas chromatograph GC 6890N of Agilent Technologies Inc. with an thermal conductivity detector. Gas phase concentration of oxygen was measured by a trace oxygen dipping probe from PreSens Precision Sensing GmbH. Oxygen concentration was measured by fluorescence quenching, whereas the degree of quenching correlates to the partial pressure of oxygen in the gas phase. Oxygen measurement indicated a concentration of 0.1% vol of O₂ in the used synthesis gas.

During the experiment samples of 5 mL were taken for the determination of OD₆₀₀, pH and product concentrations. The latter were determined by quantitative ¹H-NMR-spectroscopy.

After inoculation of C. ljungdahlii, cells began to grow with a growth rate p of 0.062 h⁻¹ and continuously produced acetate up to a concentration of 6.2 g/L after 94.5 hours. Concomitant to the production of acetate, ethanol was produced in a lower rate compared to the production of acetate up to a concentration of 1 g/L after 94.5 hours.

TABLE 1
Results of Example 6

NMR-analytics

| Process | | | Acetate, | Ethanol, |
|---|---|---|---|---|
| time, h | pH | OD600 | mg/L | mg/L |
| 0.0 | 6.15 | 0.10 | 18 | n.d. |
| 18.0 | 5.97 | 0.69 | 973 | 97 |
| 42.5 | 5.20 | 1.50 | | |
| 66.0 | 4.67 | 1.95 | 5368 | 966 |
| 94.5 | 4.54 | 1.77 | 6187 | 1070 |

(n.d. = not detected)

### Example 7

Oxidation of Butanol to Butyric Acid with Escherichia coli Starting from Clostridium autoethanogenum Chemolithoautotrophic Ethanol/Acetate Production Medium

The homoacetogenic bacterium Clostridium autoethanogenum was cultivated on synthesis gas for the biotransformation of hydrogen and carbon dioxide to ethanol and acetate. All C. autoethanogenum cultivation steps were carried out under anaerobic conditions in pressure-resistant glass bottles that can be closed airtight with a butyl rubber stopper.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7H₂O; 0.02 g/L CaCl₂×2H₂O; 20 mg/L nitrilotriacetic acid; 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochlorid and 400 mg/L Na₂S×9H₂O were inoculated with 5 mL of a frozen cryo stock of C. autoethanogenum.

The chemolithoautotrophic cultivation was carried out in a 1 L pressure-resistant glass bottle at 37° C., 150 rpm and a ventilation rate of 1 L/h with a premixed gas with 67% Hz, 33% CO₂ in an open water bath shaker for 70.3 h. The gas was discharged into the medium through a sparger with a pore size of 10 μm, which was mounted in the center of the reactors. Culturing was carried out without pH control.

After precultivation in ATCC1754-medium, the cells were transferred to a first chemolithoautotrophic production culture. Therefore ⅓ of the preculture suspension was centrifuged (10 min, 3234×g, room temperature) and the pellet was resuspended in 500 ml LM33 mineral medium (pH=4.25, 1.3 g/L KOH, 0.5 g/L MgCl₂, 0.21 g/L NaCl, 0.135 g/L CaCl₂×2H₂O, 2.65 g/L NaH₂PO₄×2H₂O, 0.5 g/L KCl, 2.5 g/L NH₄Cl, 15 mg/L nitrilotriacetic acid, 30 mg/L MgSO₄×7H₂O, 5 mg/L MnSO₄×H₂O, 1 mg/L FeSO₄×7H₂O, 8 mg/L Fe(SO₄)₂(NH₄)₂×6H₂O, 2 mg/L CoCl₂×6H₂O, 2 mg/L ZnSO₄×7H₂O, 200 μg/L CuCl₂×2H₂O, 200 μg/L KAI(SO₄)₂×12H₂O, 3 mg/L H₃BO₃, 300 μg/L Na₂MoO₄×2H₂O, 200 μg/L Na₂SeO₃, 200 μg/L NiCl₂×6H₂O, 200 μg/L Na₂WO₄×6H₂O, 200 μg/L d-biotin, 200 μg/L folic acid, 100 μg/L pyridoxine-HCl, 500 μg/L thiamine-HCl; 500 μg/L riboflavin; 500 μg/L nicotinic acid; 500 μg/L Ca-pantothenate; 500 μg/L vitamin B12; 500 μg/L p-aminobenzoate; 500 μg/L lipoic acid, 10 mg/L FeCl₃, aerated for 30 min with a premixed gas with 67% Hz and 33% CO₂) with additional 500 mg/L L-cysteine-hydrochlorid and 0.5 mg/L resazurin. The pH was adjusted to 5.8 before the addition of the cells and hold constantly at this level by automatic addition of 100 g/L NaOH solution by a Titrino pH control system (Methrom, Switzerland). The production was carried out in a 1 L pressure-resistant glass bottle at 37° C., 150 rpm and a ventilation rate of 1 L/h with a premixed gas with 67% Hz, 33% CO₂ in an open water bath shaker for 93.5 h. The gas was discharged into the medium through a sparger with a pore size of 10 μm, which was mounted in the center of the reactors. At the start and during the incubation time, samples were taken to record optical density, pH and different analytes. The analyte determination was performed by semiquantitative ¹H-NMR spectroscopy. As an internal quantification standard sodium trimethylsilylpropionate (T(M)SP) was used.

During the cultivation the concentration of ethanol increased from 0 g/L to 0.5 g/L and the acetate concentration increased from 0 g/L to 4.25 g/L. The production culture was started with an OD_{600 nm} of 0.084 and stopped after 93 h at an OD_{600 nm} of 0.433.

The production culture was afterwards harvested (10 min, 3234×g, room temperature) and transferred to fresh LM33-medium for a second production culture. The cell pellet was resuspended in 500 ml LM33 mineral (aerated for 30 min with a premixed gas with 67% Hz and 33% CO₂) with additional 500 mg/L L-cysteine-hydrochlorid and 0.5 mg/L resazurin. The pH was adjusted to 5.8 before the addition of the cells and hold constantly at this level by automatic addition of 100 g/L NaOH solution by a Titrino pH control system. The production was carried out in a 1 L pressure-resistant glass bottle at 37° C., 150 rpm and a ventilation rate of 1 L/h with a premixed gas with 67% Hz, 33% CO₂ in an open water bath shaker for 90.5 h. The gas was discharged into the medium through a sparger with a pore size of 10 μm, which was mounted in the center of the reactors.

At the start and during the incubation time, samples were taken to record optical density, pH and different analytes. The production culture was started with an OD_{600 nm} of 0.106, reached 0.55 after 65 h and decreased to an OD_{600 nm} of 0.435 when the production was stopped after 90.5 h. During the production the concentration of ethanol increased from 0 g/L to 0.5 g/L and the acetate concentration increased from 0 g/L to 13.3 g/L.

Strain E. coli W3110 ΔfadE harboring the plasmid pBT10 was applied for the biotransformation of butanol to butyrate. The plasmid pBT10 is described in WO2009/077461 and the E. coli strain is described in WO2013/092547. All E. coli cultivations were carried out at ambient atmosphere. Strain E. coli W3110 ΔfadE pBT10 was cultivated on plate count agar (Merck, Germany) with 50 mg/l kanamycin. For a first preculture 25 mL of LB medium (Merck, Germany) with 50 mg/L kanamycin in a 250 mL shaking flask were inoculated with a single colony from a fresh incubated agar plate and cultivated at 37° C. and 200 rpm for 16 h. For a second preculture 100 mL of HZD medium (1.8 g/L (NH₄)₂SO₄, 19.1 g/L K₂HPO₄, 12.5 g/L KH₂PO₄, 6.7 g/L yeast extract, 2.3 g/L Na₃-Citrat*2H₂O, 170 mg/L NH₄Fe-Citrat, 5 mL/L trace elements US3 (80 mL/L 37% HCl, 1.9 g/L MnCl₂*4H₂O, 1.9 g/L ZnSO₄*7H₂O, 0.9 g/L Na-EDTA*2H₂O, 0.3 g/l H₃BO₃, 0.3 g/L Na₂MoO₄*2H₂O, 4.7 g/L CaCl₂*2H₂O, 17.8 g/L FeSO₄*7H₂O, 0.2 g/L CuCl₂*2H₂O), 30 mL/L HZD-feed (50 g/kg glucose×H₂O, 10 g/kg MgSO₄×7H₂O, 22 g/kg NH₄Cl)) with 50 mg/L kanamycin in a 1000 mL shaking flask were inoculated with OD_{600 nm} of 0.1 from the first preculture and cultivated at 37° C. and 200 rpm for 8 h.

For the main culture 15 L of fresh HZD medium (pH 6.8) with 15 g/L glucose in a 20 L stirred tank bioreactor were inoculated with cells from the second preculture to an OD_{600 nm} of 0.1. The fermentation was carried out at 37° C. and 30% pO₂ (250 to 1200 rpm with an airflow of 4.2-30 L/min). The pH was hold at 6.8 with 25% NH₄OH. When the pO₂ reached 45% a feed with 5 g/L*h glucose was started. 4 h before harvest the culture was induced with 0.025% DCPK. After harvest at high OD_{600 nm} the cells were centrifuged and stored at −20° C.

The deep-freezed cells of the recombinant E. coli W3110 ΔfadE pBT10 were thawed on ice and resuspended in 16.5 ml assay buffer (pH 7.34, 1.347 g/L KH₂PO₄, 6.98 g/L K₂HPO₄, 0.5 g/L NH₄Cl) to an OD_{600 nm} of 4.5 including 1 g/L butanol as substrate and 9.9% (v/v) filter-sterilized supernatant of the second production culture of C. autoethanogenum as co-substrate for the butanol oxidation reaction. The reaction was carried out in a 50 mL reaction tube at 30° C. and 300 rpm in a water bath shaker for 30 h.

At the start and during the incubation time, samples were taken to record optical density, pH and different analytes. The determination of the product concentrations was performed by semiquantitative ¹H-NMR spectroscopy. As an internal quantification standard sodium trimethylsilylpropionate (T(M)SP) was used.

During 6 h of biotransformation the concentration of acetate decreased from 1.35 g/L to 0 g/L. The concentration of butanol decreased from 0.95 g/L to 0 g/L and the concentration of butyrate increased from 0 g/L to 1.1 g/L.

### Example 8

Oxidation of Butanol to Butyric Acid with Escherichia coli and Acetate as Co-Substrate

Strain E. coli W3110 ΔfadE harboring the plasmid pBT10 was applied for the biotransformation of butanol to butyrate. The plasmid pBT10 is described in WO2009/077461 and the E. coli strain is described in WO2013/092547.

Strain E. coli W3110 ΔfadE pBT10 was cultivated on plate count agar (Merck, Germany) with 50 mg/l kanamycin. For a first preculture 25 mL of LB medium (Merck, Germany) with 50 mg/L kanamycin in a 250 mL shaking flask were inoculated with a single colony from a fresh incubated agar plate and cultivated at 37° C. and 200 rpm for 16 h. For a second preculture 100 mL of HZD medium (1.8 g/L (NH₄)₂SO₄, 19.1 g/L K₂HPO₄, 12.5 g/L KH₂PO₄, 6.7 g/L yeast extract, 2.3 g/L Na₃-Citrate*2H₂O, 170 mg/L NH₄Fe-Citrate, 5 mL/L trace elements US3 (80 mL/L 37% HCl, 1.9 g/L MnCl₂*4H₂O, 1.9 g/L ZnSO₄*7H₂O, 0.9 g/L Na-EDTA*2H₂O, 0.3 g/l H₃BO₃, 0.3 g/L Na₂MoO₄*2H₂O, 4.7 g/L CaCl₂*2H₂O, 17.8 g/L FeSO₄*7H₂O, 0.2 g/L CuCl₂*2H₂O), 30 mL/L HZD-feed (50 g/kg glucose×H₂O, 10 g/kg MgSO₄×7H₂O, 22 g/kg NH₄Cl)) with 50 mg/L kanamycin in a 1000 mL shaking flask were inoculated with OD_{600 nm} of 0.1 from the first preculture and cultivated at 37° C. and 200 rpm for 8 h.

For the main culture 15 L of fresh HZD medium (pH 6.8) with 15 g/L glucose in a 20 L stirred tank bioreactor were inoculated with cells from the second preculture to an OD_{600 nm} of 0.1. The fermentation was carried out at 37° C. and 30% pO₂ (250 to 1200 rpm with an airflow of 4.2-30 L/min). The pH was hold at 6.8 with 25% NH₄OH. When the pO₂ reached 45% a feed with 5 g/L*h glucose was started. 4 h before harvest the culture was induced with 0.025% DCPK. After harvest at high OD_{600 nm} the cells were centrifuged and stored at −20° C.

The deep-freezed cells of the recombinant E. coli W3110 ΔfadE pBT10 were thawed on ice and resuspended in 15 ml assay buffer (pH 7.4, 1.347 g/L KH₂PO₄, 6.98 g/L K₂HPO₄, 0.5 g/L NH₄Cl) to an BTM of 1.49 g/L for each assay including 0.1853 g/L butanol as substrate and 1.723 or 0.172 g/L ammonium acetate as co-substrate. The oxidation reaction was carried out in a 50 mL reaction tube at 30° C. and 300 rpm in a water bath shaker for 3 h.

At the start and during the incubation time, samples were taken to record optical density, pH and different analytes. The determination of the product concentrations was performed by semiquantitative ¹H-NMR spectroscopy. As an internal quantification standard sodium trimethylsilylpropionate (T(M)SP) was used.

During the incubation period of 3 h the concentration of acetate decreased from 1.59 g/L to 0.68 g/L for the assay with higher acetate concentration. The concentration of butanol decreased from 174 mg/L to 0 mg/L and the concentration of butyrate increased from 0 to 122.6 mg/L. In the assay with lower acetate concentration acetate was decreased from 0.15 g/L to 0 g/L in 1 h, whereas the concentration of butanol decreased from 177 mg/L to 18 mg/L and the concentration of butyrate increased from 0 to 194 mg/L.

### Example 9

Pseudomonas putida Forming Rhamnolipids from Acetate and Decanoic Acid

For the biotransformation of acetate and decanoic acid to rhamnolipids a plasmid harboring Pseudomonas putida KT2440 strain was used. The plasmid pBBR1MCS-2::ABC is described in example 2 of DE 10 2010 032 484 A1 and the transformation of Pseudomonas putida KT2440 with the vector is described in Iwasaki et al. Biosci. Biotech. Biochem. 1994. 58(5): 851-854. The recombinant Pseudomonas putida KT2440 pBBR1MCS-2::ABC was cultivated on LB agar plates with 50 mg/l kanamycin.

For the preculture 10 ml of LB medium with 50 mg/l kanamycin in a 100 ml shaking flask were inoculated with a single colony from a fresh incubated agar plate and cultivated at 30° C. and 120 rpm for 15 h to an OD_{600 nm}>3.5. Then the cell suspension was centrifuged, washed with fresh M9_BS_Ac medium and centrifuged again.

For the main culture 100 ml of fresh M9_BS_Ac medium (pH 7.4; 6.81 g/L Na₂HPO₄, 2.4 g/L KH₂PO₄, 0.4 g/L NaCl, 1.4 g/L NH₄Cl, 2 ml/L 1 M MgSO₄×7H₂O, 1.63 g/L ¹³C₂—Na-acetate, 0.13 ml/L 25% HCl, 1.91 mg/L MnCl₂×7H₂O, 1.87 mg/L ZnSO₄×7H₂O, 0.84 mg/L Na-EDTA×2H₂O, 0.3 mg/L H₃BO₃, 0.25 mg/L Na₂MoO₄×2H₂O, 4.7 mg/L CaCl₂×2H₂O, 17.8 mg/L FeSO₄×7H₂O, 0.15 mg/L CuCl₂×2H₂O) in a 500 ml shaking flask were inoculated with centrifuged and washed cells from the preculture to an OD_{600 nm} of 0.12. This culture was incubated at 32° C. and 140 rpm for 142 h. After 6 h of cultivation, 2 g/L rhamnose were added to the culture for induction. After 22.5 h of cultivation, 1 g/L decanoic acid was added to the culture. After 7.5 h, 22.5 h, 30.5 h, 47.25 h and 53 h of cultivation, 1 g/l ¹³C₂—Na-acetate were added respectively. At the start and during the culturing period, samples were taken. These were tested for optical density, pH and the different analytes (tested by NMR).

The results showed that in the main culture the amount of acetate decreased continuously from 1.63 g/l in the beginning to 0 g/l after 71.75 h (including the acetate feeding of 5 g/L ¹³C₂—Na-acetate). The concentration of decanoic acid decreased from 1 g/l at 22.5 h to 0 g/L after 71.75 h. Also, the concentration of rhamnolipids (2RL-C10-C10), a dirhamnosyl lipid was increased from 0.0 mg/l to 779 mg/l after 71.75 h of cultivation. The newly formed rhamnolipids were ¹³C-labeled (34% in the fatty acid part). The carbon yield for ¹³C-labeled 2RL-C10-C10, the dirhamnosyl lipid was about 6.05% based on the consumed acetate and decanoic acid and for non-labeled 2RL-C10-C10 it was 11.75%. This showed that a larger percentage of the resulting rhamnolipids were formed from the unlabeled decanoic acid than the acetate.

### Example 10

Pseudomonas putida Forming Rhamnolipids from Acetate and Hexanoic Acid

For the biotransformation of acetate and hexanoic acid to rhamnolipids a plasmid harboring Pseudomonas putida KT2440 strain is used. The plasmid pBBR1MCS-2::ABC is described in example 2 of DE 10 2010 032 484 A1 and the transformation of Pseudomonas putida KT2440 with the vector is described in Iwasaki et al. Biosci. Biotech. Biochem. 1994. 58(5): 851-854. The recombinant Pseudomonas putida KT2440 pBBR1MCS-2::ABC is cultivated on LB agar plates with 50 mg/l kanamycin. For the preculture 10 ml of LB medium with 50 mg/l kanamycin in a 100 ml shaking flask are inoculated with a single colony from a fresh incubated agar plate and cultivated at 30° C. and 120 rpm for 15 h to an OD_{600 nm}>3.5. Then the cell suspension is centrifuged, washed with fresh M9_BS_Ac medium and centrifuged again.

For the main culture 100 ml of fresh M9_BS_Ac medium (pH 7.4; 6.81 g/L Na₂HPO₄, 2.4 g/L KH₂PO₄, 0.4 g/L NaCl, 1.4 g/L NH₄Cl, 2 ml/L 1 M MgSO₄×7H₂O, 1.63 g/L ¹³C₂—Na-acetate, 0.13 ml/L 25% HCl, 1.91 mg/L MnCl₂×7H₂O, 1.87 mg/L ZnSO₄×7H₂O, 0.84 mg/L Na-EDTA×2H₂O, 0.3 mg/L H₃BO₃, 0.25 mg/L Na₂MoO₄×2H₂O, 4.7 mg/L CaCl₂×2H₂O, 17.8 mg/L FeSO₄×7H₂O, 0.15 mg/L CuCl₂×2H₂O) in a 500 ml shaking flask are inoculated with centrifuged and washed cells from the preculture to an OD_{600 nm} of 0.12. This culture is incubated at 32° C. and 140 rpm for 142 h. After 6 h of cultivation, 2 g/L rhamnose are added to the culture for induction. After 22.5 h of cultivation, 1 g/L hexanoic acid is added to the culture. After 7.5 h, 22.5 h, 30.5 h, 47.25 h and 53 h of cultivation, 1 g/l ¹³C₂—Na-acetate are added respectively. At the start and during the culturing period, samples are taken. These are tested for optical density, pH and the different analytes (tested by NMR).

In the main culture the amount of acetate decreases continuously to 0 g/l after 71.75 h (including the acetate feeding of 5 g/L ¹³C₂—Na-acetate). The concentration of hexanoic acid also decreases to 0 g/L after 71.75 h. Also, the concentration of rhamnolipid (2RL-C10-C10) increases during the cultivation. The newly formed rhamnolipids were ¹³C-labeled (<80% in the fatty acid part). The carbon yield for ¹³C-labeled 2RL-C10-C10, a dirhamnosyl lipid related to consumed acetate and hexanoic acid is lower and for non-labeled 2RL-C10-C10 it is higher than in cultures without hexanoic acid feeding. Again this confirms the finding that a larger percentage of the resulting rhamnolipids is formed from the unlabeled hexanoic acid than the acetate.

### Example 11

Oxidation of Dodecane with Escherichia coli and Acetate as Co-Substrate

For the oxidation of dodecane the plasmid harboring strain E. coli W3110 ΔfadE ΔbioH pBT10_alkL was used. The construction of plasmid pBT10_alkL is described in example 1 of WO/2011/131420

(SEQ ID NO: 8) and the mutations of the E. coli strain are described in EP12007663 (ΔbioH) and EP2744819 (ΔfadE). For a first preculture 25 mL of LB medium (Merck, Germany) supplemented with 50 mg/L kanamycin in a 250 mL shaking flask were inoculated with frozen cell material from a cryoculture and cultivated at 37° C. and 200 rpm for 16 h.

For a second preculture 100 mL of HZD medium (1.8 g/L (NH4)2SO4, 19.1 g/L K2HPO4, 12.5 g/L KH2PO4, 6.7 g/L yeast extract, 2.3 g/L Na3-Citrat*2H2O, 170 mg/L NH4Fe-Citrat, 5 mL/L trace elements US3 (40 mL/L 37% HCl, 1.9 g/L MnCl2*4H2O, 1.9 g/L ZnSO4*7H2O, 0.9 g/L Na-EDTA*2H2O, 0.3 g/l H3BO3, 0.3 g/L Na2MoO4*2H2O, 4.7 g/L CaCl2*2H2O, 17.8 g/L FeSO4*7H2O, 0.2 g/L CuCl2*2H₂O), 30 mL/L HZD-feed (550 g/L glucose×H2O, 10 g/L MgSO4×7H2O, 22 g/L NH4Cl)) with 50 mg/L kanamycin in a 1000 mL shaking flask were inoculated with an OD_{600 nm} of 0.2 with the first preculture and cultivated at 37° C. and 200 rpm for 7 h. For cryoconservation the whole culture was mixed with 99%-glycerine (to an end-concentration of 10% (w/w) glycerine) and subdivided into cryotubes, each volume to inoculate one main culture with an OD of 0.3. These aliquots were stored at 80° C.

For the main culture 100 ml of HZD medium with 50 mg/L kanamycin in a 1000 ml shaking flask were inoculated with one frozen cell aliquot and cultivated at 37° C. and 180 rpm. After 2.5 h the temperature was shifted to 25° C. and after 3 h of cultivation the culture was induced by addition of 0.005% (v/v) DCPK (dicyclopropylketone, Merck). The cells were harvested after 19 h of cultivation and directly used for the oxidation reaction.

For the oxidation reaction 35 mL assay buffer (200 mM potassium phosphate buffer, pH 6.8; 13.77 g/L KH¬2 PO4, 17.22 g/L K2HPO4, 0.5 g/L NH4Cl and 1.72 g/l potassium acetate as co-substrate) with 50 mg/L kanamycin in a 250 mL pressure resistant bottle were inoculated with cells from the main culture to an OD_{600 nm} of 11. The culture was supplemented with 18 mL dodecane (ABCR) and incubated at 30° C., 200 rpm and surface-aerated with 1 L/h synthetic air (20% O₂, 80% N2, Linde) in an open water bath shaker for 23 h.

At the start and during incubation period, pH and OD measurements were performed from samples of the aqueous phase. From both, the aqueous and the organic phase, samples were taken and analyzed by Cedex analytics (for acetate quantification) an LCMS analytics (for dodecane oxidation products).

During the incubation period the cosubstrate acetate decreased from 1.74 g/L to 0 g/L. The 1-dodecanol concentration increased from 0 μg/L to 150.67 μg/L, the 1-dodecanoic acid concentration increased from 0 μg/L to 333.83 μg/L, the 12-hydroxydodecanoic acid concentration increased from 0 μg/L to 18.3 μg/L, the oxododecanoic acid concentration increased from 0 μg/L to 1.52 μg/L and the 1,12-didodecanoic acid concentration increased from 0 μg/L to 189.06 μg/L.

1. A method of oxidising at least one organic substance in aerobic conditions to produce at least one alcohol, amine, acid, aldehyde, rhamnolipid and/or ketone, the method comprising:
(a) producing ethanol and/or acetate from a carbon source in aerobic conditions, comprising
(i) contacting the carbon source with a reaction mixture comprising
a first acetogenic microorganism in an exponential growth phase;
free oxygen; and
a second acetogenic microorganism in a stationary phase
wherein the first and second acetogenic microorganism is capable of converting the carbon source to the acetate and/or ethanol; and
(b) contacting the acetate and/or ethanol from step (a) with the organic substance and with a third microorganism capable of oxidising the organic substance to produce the alcohol, amine, acid, aldehyde, rhamnolipid and/or ketone and
wherein the acetate is a co-substrate.

2. The method according to claim 1, organic substance is selected from the group consisting of branched or unbranched, saturated or unsaturated, optionally substituted alkanes, alkenes, alkynes, alcohols, aldehydes, ketones, carboxylic acids, esters of carboxylic acids, amines and epoxides.

3. The method according to claim 1, wherein the acetate concentration is at least 10 ppm in step (b), preferably 100 ppm.

4. The method according to claim 1, wherein the organic compound is:
(a) an alkane oxidised in step (b) to form the corresponding alcohol;
(b) an alcohol oxidised in step (b) to form the corresponding amine, acid, aldehyde, and/or ketone;
(c) a pyruvate oxidised in step (b) to form acetate
(d) a carboxylic acid oxidised in step (b) to form the corresponding alkene or rhamnolipid and/or
(e) an aldehyde oxidised in step (b) to form the corresponding carboxylic acid.

5. The method according to claim 1, wherein the third microorganism is genetically modified to increase expression of at least one oxidising enzyme relative to the wild type cell, wherein the oxidising enzyme is selected from the group consisting of alkane monooxygenase, a xylene monooxygenase, an aldehyde dehydrogenase, an alcohol oxidase and an alcohol dehydrogenase.

6. The method according to claim 5, wherein,
(a) the alkane monooxygenase is a cytochrome-P450 monooxygenase;
(b) the alkane monooxygenase is an alkB gene product which is encoded by an alkB gene from at least one Gram-negative bacteria; and/or
(c) the alcohol dehydrogenase is the alcohol dehydrogenase encoded by the alkJ gene from at least one Gram-negative bacteria.

7. The method according to claim 6, wherein the Gram-negative bacteria is selected from the group consisting of Pseudomonads, Azotobacter, Desulfitobacterium, Burkholderia, Xanthomonas, Rhodobacter, Ralstonia, Delftia, Rickettsia, Oceanicaulis, Caulobacter, Marinobacter, and Rhodopseudomonas.

8. The method according to claim 6, wherein the alkL gene product comprises an amino acid sequence selected from the group consisting of SEQ ID Nos: 1-4.

9. The method according to claim 1, wherein the first and second microorganism is selected from the group consisting of Clostridium autothenogenum DSMZ 19630, Clostridium ragsdahlei ATCC no. BAA-622, Clostridium autoethanogenum, Moorella sp HUC22-1, Moorella thermoaceticum, Moorella thermoautotrophica, Rumicoccus productus, Acetoanaerobum, Oxobacter pfennigii, Methanosarcina barkeri, Methanosarcina acetivorans, Carboxydothermus, Desulfotomaculum kutznetsovii, Pyrococcus, Peptostreptococcus, Butyribacterium methylotrophicum ATCC 33266, Clostridium formicoaceticum, Clostridium butyricum, Lactobacillus delbrukii, Propionibacterium acidoproprionici, Proprionispera arboris, Anaeroblerspirillum succiniproducens, Bacterioides amylophilus, Becterioides ruminicola, Thermoanaerobacter kivui, Acetobacterium woodii, Acetoanaerobium notera, Clostridium aceticum, Butyribacterium methylotrophicum, Moorella thermoacetica, Eubacterium limosum, Peptostreptococcus productus, Clostridium ljungdahlii, Clostridium ATCC 29797 and Clostridium carboxidivorans.

10. The method according to claim 1, wherein the third organism is selected from the group consisting of E. coli, Pseudomonas sp., Pseudomonas fluorescens, Pseudomonas putida, Pseudomonas acidovorans, Pseudomonas aeruginosa, Acidovorax sp., Acidovorax temperans, Acinetobacter sp., Burkholderia sp., cyanobacteria, Kiebsiella sp., Salmonella sp., Rhizobium sp. and Rhizobium meliloti.

11. The method according to claim 1, wherein the first and/or second microorganism is Clostridium ljungdahlii and the third microorganism is Escherichia coli.

12. The method according to claim 1, wherein the first acetcgenic microorganism in the exponential growth phase has a growth rate of 0.01 to 2 h⁻¹ and/or an OD₆₀₀ of 0.01 to 2.

13. The method according to claim 1, wherein the aerobic conditions is a result of oxygen being at a concentration of 0.000005-1% volume in the gas phase.

14. The method according to claim 1, wherein the carbon source comprises CO.

15. The method according to claim 1, wherein steps (a) and (b) are carried out in separate fermenters.
